(19) United States
(12) Patent Application Publication
(10) Pub. No.: US 2026/0144730 A1
(43) Pub. Date: May 28, 2026

(54) COMPOSITION FOR SKIN ANTI-AGING, SKIN BRIGHTENING OR SKIN REVERSE-AGING CONTAINING AMIDE-BASED COMPOUND

(71) Applicant: AMOREPACIFIC CORPORATION, Seoul (KR)

(72) Inventors: Hyunjung CHOI, Yongin-si (KR); Geunhyuk JANG, Yongin-si (KR); Eun Jeong CHOI, Yongin-si (KR); Hyoung June KIM, Yongin-si (KR); Wonseok PARK, Yongin-si (KR); Heungsoo BAEK, Yongin-si (KR); Jaewon YOU, Yongin-si (KR)

(73) Assignee: AMOREPACIFIC CORPORATION, Seoul (KR)

(21) Appl. No.: 19/311,346

(22) Filed: Aug. 27, 2025

(30) Foreign Application Priority Data
Aug. 28, 2024 (KR) 10-2024-0116067
Aug. 12, 2025 (KR) 10-2025-0111632

(51) Int. Cl. A61K 8/49; A61Q 19/02; A61Q 19/08

(52) U.S. Cl. CPC A61K 8/4933; A61Q 19/02; A61Q 19/08; A61K 2800/92

(57) ABSTRACT

The present specification relates to a composition including a new amide-based compound derived from an amino acid structure such as valine, leucine, phenylalanine, proline, pipecolic acid, or the like, wherein the composition exhibits a skin anti-aging or skin reverse-aging effect by restoring the size and number of dendrites of aged melanin-producing cells to those of young cells. As a result, the composition may exhibit a skin brightening effect by inhibiting the amount of melanin produced in the melanin-producing cells.

### CROSS REFERENCE TO RELATED APPLICATIONS

The present application claims priority to Korean Patent Application No. 10-2024-0116067, filed Aug. 28, 2024, and Korean Patent Application No. 10-2025-0111632, filed Aug. 12, 2025, the entire contents of which are hereby incorporated by this reference.

### BACKGROUND OF THE INVENTION

### Field of the Invention

The present specification discloses a composition for skin anti-aging, skin brightening, or skin reverse-aging, comprising an amide-based compound.

### Description of the Related Art

Melanin-producing cells present in human skin may cause senile pigmentation by excessively producing melanin when aged by light.

Specifically, when the skin is exposed to ultraviolet rays, melanin is produced for skin protection, and when ultraviolet rays are absent, the skin returns to its original state. However, as age increases, even in the absence of ultraviolet rays, the skin continuously produces an excessive amount of melanin due to the memory of ultraviolet rays previously received, thereby causing hyperpigmentation such as freckles and senile lentigo.

Conventional skin tone care substances for solving this problem mainly appeal to preventive effects. Therefore, there are not many functional materials having the concept of treatment that can treat skin pigmentation already caused by ultraviolet rays or inflammation. However, in order to inhibit skin hyperpigmentation, both prevention and treatment must be performed in parallel.

### SUMMARY OF THE INVENTION

In one aspect of the present disclosure, there is provided a composition for skin anti-aging, skin brightening, or skin reverse-aging, the composition comprising a new compound derived from an amino acid structure such as valine, leucine, phenylalanine, proline, pipecolic acid, or the like.

In one aspect, the present specification provides a composition for skin anti-aging, skin brightening, or skin reverse-aging, including as an active ingredient a compound represented by Chemical Formula 1 below, a stereoisomer thereof, a pharmaceutically acceptable salt thereof, a hydrate thereof, or a solvate thereof,

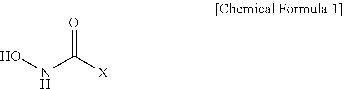

in Chemical Formula 1,
X is represented by any one of Chemical Formulae 1-1 to 1-5 below,

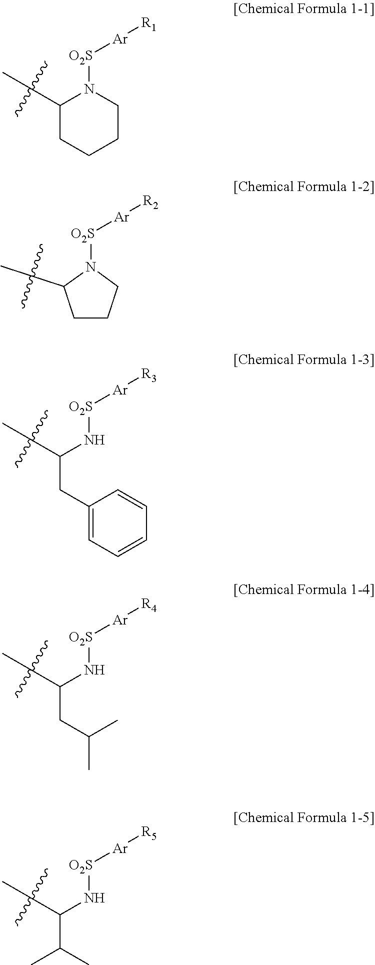

in Chemical Formula 1-1,
Ar is a phenyl which is unsubstituted or substituted with one to three R₁ groups,
R₁ is hydrogen, phenyl group, C1-C4 alkoxy group, C1-C3 alkyl group, fluoro group, adamantyl group, acetylamino group, or hydroxyiminoethyl group, and
when the number of R₁ residues is more than one, all R₁ residues are identical to each other,
in Chemical Formula 1-2,
Ar is a phenyl substituted with one R₂, and
R₂ is C1-C4 alkyl group, C1-C4 alkoxy group, adamantyl group, acetyl group, or acetylamino group,
in Chemical Formula 1-3,
Ar is a phenyl substituted with one to three R₃ groups or an unsubstituted naphthalene,
R₃ is hydrogen or a C1-C4 alkyl group, and
when the number of R₃ residues is more than one, all R₃ residues are identical to each other, and preferably, R₃ is a C1-C4 alkyl group,
in Chemical Formula 1-4,
Ar is a phenyl which is unsubstituted or substituted with one to three R₄ groups,
R₄ is hydrogen, phenyl group, C1-C4 alkyl group, or C1-C4 alkoxy group, and
when the number of R₄ residues is more than one, all R₄ residues are identical to each other, and
in Chemical Formula 1-5,
Ar is a phenyl substituted with one to three R₅ groups,
R₅ is a C1-C4 alkyl group, and
when the number of R₅ residues is more than one, all R₅ residues are identical to each other.

In one aspect, a composition comprising a new amide-based compound disclosed in the present specification may exhibit a reverse-aging effect by restoring the size and number of dendrites of aged melanin-producing cells to those of young cells, and, as a result, may exhibit a skin-brightening effect by inhibiting the amount of melanin produced in the melanin-producing cells.

### BRIEF DESCRIPTION OF THE DRAWINGS

FIGS. 1A and 1B show results of observing a cellular anti-aging effect of a composition according to one embodiment of the present disclosure.

FIGS. 2A and 2B show results of observing a melanin production inhibitory effect of a composition according to one embodiment of the present disclosure.

FIGS. 3A and 3B show results of observing a gene expression inhibitory effect of melanin production enzyme (TRP2) and a melanin decomposition effect of a composition according to one embodiment of the present disclosure.

FIGS. 4A and 4B show results of observing a gene expression inhibitory effect of an aging gene (p21) and a gene expression inhibitory effect of an aging gene (p16) of a composition according to one embodiment of the present disclosure.

FIG. 5 shows results of observing a melanin decomposition effect of a composition according to one embodiment of the present disclosure.

FIG. 6 shows results of observing a gene expression inhibitory effect of melanin production enzyme (TRP2) of a composition according to one embodiment of the present disclosure.

FIG. 7 shows results of observing a gene expression inhibitory effect of an aging gene (p16) of a composition according to one embodiment of the present disclosure.

### DETAILED DESCRIPTION OF THE INVENTION

Hereinafter, the present disclosure will be described in more detail with reference to the following embodiments. However, the following embodiments are provided for illustrative purposes only to assist in understanding the present disclosure and are not intended to limit the scope and range of the present disclosure.

In exemplary embodiments of the present disclosure, a composition for skin anti-aging, skin brightening, or skin reverse-aging, including as an active ingredient a compound represented by Chemical Formula 1 below, a stereoisomer thereof, a pharmaceutically acceptable salt thereof, a hydrate thereof, or a solvate thereof, is provided,

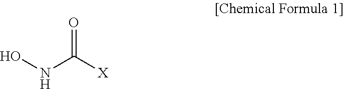

in Chemical Formula 1,
X is represented by any one of Chemical Formulae 1-1 to 1-5 below,

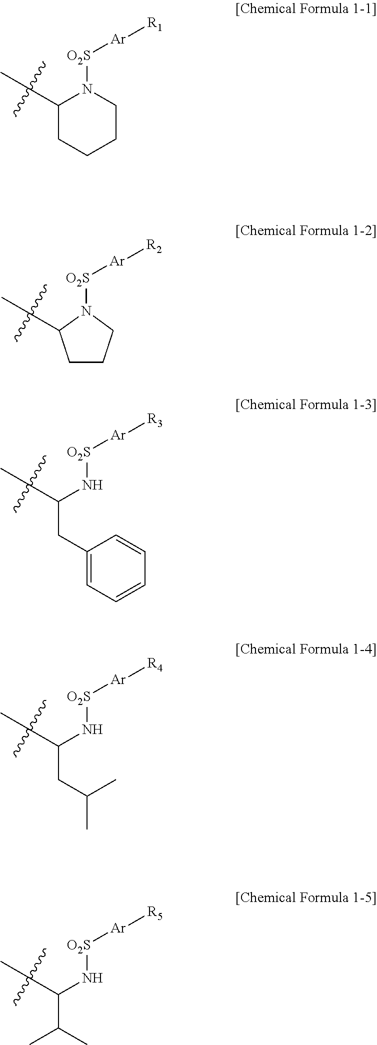

in Chemical Formula 1-1,
Ar is a phenyl which is unsubstituted or substituted with one to three Ri groups,
R₁ is hydrogen, phenyl group, C1-C4 alkoxy group, C1-C3 alkyl group, fluoro group, adamantyl group, acetylamino group, or hydroxyiminoethyl group, and
when the number of R₁ residues is more than one, all R₁ residues are identical to each other,
in Chemical Formula 1-2,
Ar is a phenyl substituted with one R₂, and
R₂ is C1-C4 alkyl group, C1-C4 alkoxy group, adamantyl group, acetyl group, or acetylamino group,
in Chemical Formula 1-3,
Ar is a phenyl substituted with one to three R₃ groups or an unsubstituted naphthalene,
R₃ is hydrogen or a C1-C4 alkyl group, and
when the number of R₃ residues is more than one, all R₃ residues are identical to each other, and preferably, R₃ is a C1-C4 alkyl group,
in Chemical Formula 1-4,
Ar is a phenyl which is unsubstituted or substituted with one to three R₄ groups,
R₄ is hydrogen, phenyl group, C1-C4 alkyl group, or C1-C4 alkoxy group, and
when the number of R₄ residues is more than one, all R₄ residues are identical to each other, and
in Chemical Formula 1-5,
Ar is a phenyl substituted with one to three R₅ groups,
R₅ is a C1-C4 alkyl group, and
when the number of R₅ residues is more than one, all R₅ residues are identical to each other.

For example, in Chemical Formulae 1-1 to 1-5, each of R₁ to R₅ may be substituted at a para-position of Ar.

In the present specification, the alkyl group may be a straight chain or a branched chain, and, for example, may be methyl, ethyl, propyl, n-propyl, isopropyl, butyl, n-butyl, isobutyl, tert-butyl, or sec-butyl.

In the present specification, the alkoxy group may be a straight chain, a branched chain, or a cyclic chain, and, for example, may be methoxy, ethoxy, n-propoxy, isopropoxy, i-propyloxy, n-butoxy, isobutoxy, tert-butoxy, or sec-butoxy.

In the present specification, the term “isomer” includes not only optical isomers (for example, essentially pure enantiomers, essentially pure diastereomers, or mixtures thereof), but also conformational isomers (that is, isomers differing only in the angles of one or more chemical bonds), positional isomers (especially tautomers), or geometric isomers (for example, cis-trans isomers).

In the present specification, the term “essentially pure” means, for example, when used in relation to enantiomers or diastereomers, that a specific compound exemplifying the enantiomer or diastereomer is present in an amount of about 90% or more, preferably about 95% or more, more preferably about 97% or more or about 98% or more, still more preferably about 99% or more, and most preferably about 99.5% or more (w/w).

In the present specification, the term “pharmaceutically acceptable” means that the substance can be used for animals, and more specifically for humans, by avoiding significant toxic effects when used at a conventional medicinal dosage, and that the substance can obtain or has obtained approval from a governmental or equivalent regulatory body, or is listed in a pharmacopoeia or otherwise recognized as a standard pharmacopoeial substance.

In the present specification, the term “pharmaceutically acceptable salt” refers to a salt according to one aspect of the present disclosure, which is pharmaceutically acceptable and has desired pharmacological activity of the parent compound. The salt may include: (1) an acid addition salt formed with an inorganic acid such as hydrochloric acid, hydrobromic acid, sulfuric acid, nitric acid, or phosphoric acid; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]oct-2-ene-1-carboxylic acid, gluconic acid, glucoheptonic acid, 3-phenylpropionic acid, trimethylacetic acid, tert-butylacetic acid, lauryl sulfuric acid, glutamic acid, hydroxynaphthoic acid, salicylic acid, stearic acid, or muconic acid; or (2) a salt formed when an acidic proton present in the parent compound is substituted.

In the present specification, the term “hydrate” means a compound in which water is bound, and is a broad concept including an inclusion compound in which no chemical bonding force exists between water and the compound.

In the present specification, the term “solvate” means a higher-order compound formed between molecules or ions of a solute and molecules or ions of a solvent.

In other exemplary embodiments of the present disclosure, there is provided a method for skin anti-aging, skin brightening, or skin reverse-aging, comprising administering to a subject in need thereof an effective amount of a composition including, as an active ingredient, a compound represented by Chemical Formula 1 described above, a stereoisomer thereof, a pharmaceutically acceptable salt thereof, a hydrate thereof, or a solvate thereof.

In other exemplary embodiments of the present disclosure, there is provided a use of a compound represented by Chemical Formula 1 described above, a stereoisomer thereof, a pharmaceutically acceptable salt thereof, a hydrate thereof, or a solvate thereof, for manufacturing a composition for skin anti-aging, skin brightening, or skin reverse-aging.

In other exemplary embodiments of the present disclosure, there is provided a non-therapeutic use of a compound represented by Chemical Formula 1 described above, a stereoisomer thereof, a pharmaceutically acceptable salt thereof, a hydrate thereof, or a solvate thereof, for skin anti-aging, skin brightening, or skin reverse-aging.

According to the present disclosure, when the active ingredient described above is treated, the morphology of model cells, which have been aged and thereby enlarged in size and increased in number of dendrites, may become smaller in cell size and decreased in the number of dendrites, like younger cells. In addition, the expression of aging genes may be inhibited, and the gene expression of melanin production enzymes may be inhibited, so that the excessively increased amount of melanin production may be reduced to the level of young cells.

For example, the reverse-aging composition may be a composition for skin reverse-aging or for melanin-producing cell reverse-aging.

In one embodiment, the compound represented by Chemical Formula 1 may be any one selected from the following compounds:
N-hydroxy-1-(phenylsulfonyl)piperidine-2-carboxamide,
1-([1,1′-biphenyl]-4-ylsulfonyl)-N-hydroxypiperidine-2-carboxamide,
1-((4-butoxyphenyl)sulfonyl)-N-hydroxypiperidine-2-carboxamide,
N-hydroxy-1-(mesitylsulfonyl)piperidine-2-carboxamide,
2-((4-(tert-butyl)phenyl)sulfonamido)-N-hydroxy-3-phenylpropanamide,
N-hydroxy-3-phenyl-2-((2,4,6-trimethylphenyl)sulfonamido)propanamide,
N-hydroxy-2-(naphthalen-2-sulfonamido)-3-phenylpropanamide,
2-([1,1′-biphenyl]-4-sulfonamido)-N-hydroxy-4-methylpentanamide,
2-((4-(tert-butyl)phenyl)sulfonamido)-N-hydroxy-4-methylpentanamide,
2-((4-butoxyphenyl)sulfonamido)-N-hydroxy-4-methylpentanamide,
N-hydroxy-4-methyl-2-((2,4,6-trimethylphenyl)sulfonamido)pentanamide,
2-((4-(tert-butyl)phenyl)sulfonamido)-N-hydroxy-3-methylbutanamide,
N-hydroxy-3-methyl-2-((2,4,6-trimethylphenyl)sulfonamido)butanamide,
1-((4-fluorophenyl)sulfonyl)-N-hydroxypiperidine-2-carboxamide,
1-((4-((3r,5r,7r)-adamantan-1-yl)phenyl)sulfonyl)-N-hydroxypiperidine-2-carboxamide,
N-hydroxy-1-((4-propylphenyl)sulfonyl)piperidine-2-carboxamide,
1-((4-acetamidophenyl)sulfonyl)-N-hydroxypiperidine-2-carboxamide,
1-((4-(tert-butyl)phenyl)sulfonyl)-N-hydroxypyrrolidine-2-carboxamide,
1-((4-butoxyphenyl)sulfonyl)-N-hydroxypyrrolidine-2-carboxamide,
1-((4-((3r,5r,7r)-adamantan-1-yl)phenyl)sulfonyl)-N-hydroxypyrrolidine-2-carboxamide,
N-hydroxy-1-((4-propylphenyl)sulfonyl)pyrrolidine-2-carboxamide,
1-((4-acetylphenyl)sulfonyl)-N-hydroxypyrrolidine-2-carboxamide,
N-hydroxy-1-((4-(1-(hydroxyimino)ethyl)phenyl)sulfonyl)piperidine-2-carboxamide,
N-hydroxy-4-methyl-2-(phenylsulfonamido)pentanamide, and
1-((4-acetamidophenyl)sulfonyl)-N-hydroxypyrrolidine-2-carboxamide.

In one embodiment, the compound represented by Chemical Formula 1 may be represented by any one selected from the following chemical formulae:

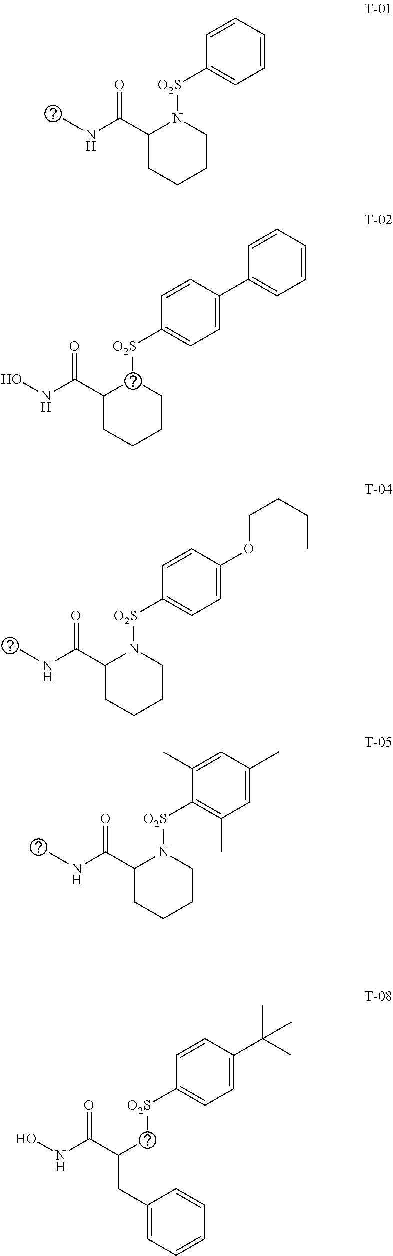

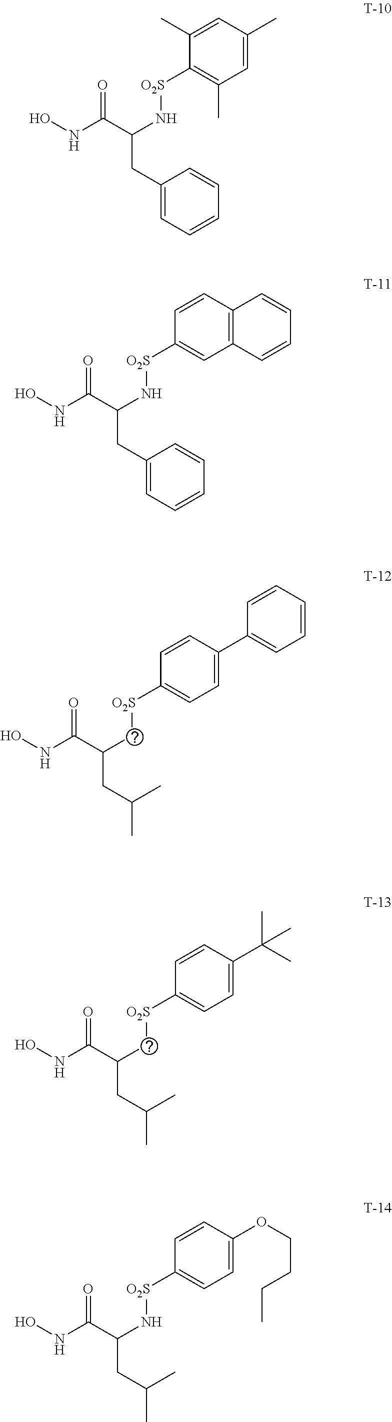 [SMILES: Cc1cc(C)c(S(=O)(=O)NC(Cc2ccccc2)C(=O)NO)c(C)c1 ; O=C(NO)C(Cc1ccccc1)NS(=O)(=O)c1ccc2ccccc2c1 ; CC(C)CC(CS(=O)(=O)c1ccc(-c2ccccc2)cc1)C(=O)NO ; CC(C)CC(CS(=O)(=O)c1ccc(C(C)(C)C)cc1)C(=O)NO ; CCCCOc1ccc(S(=O)(=O)NC(CC(C)C)C(=O)NO)cc1]

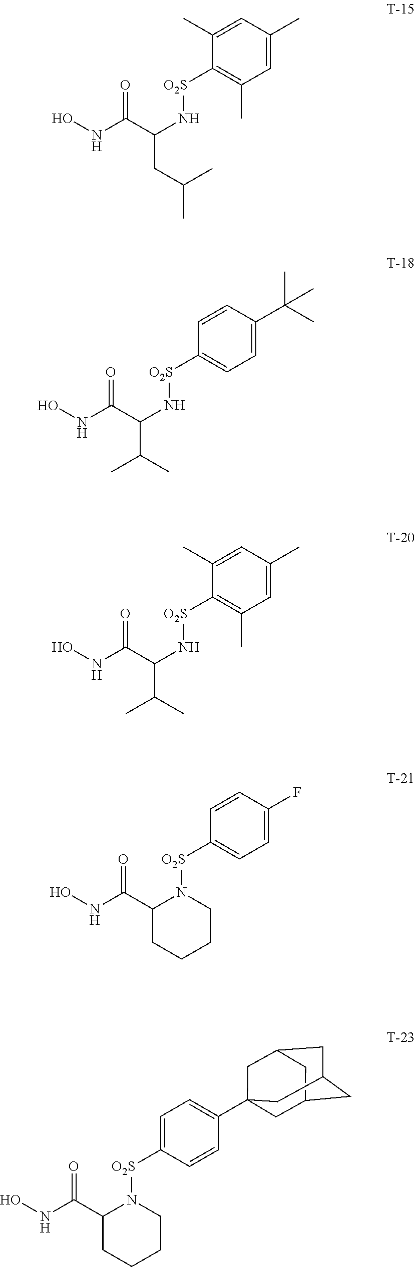 [SMILES: Cc1cc(C)c(S(=O)(=O)NC(CC(C)C)C(=O)NO)c(C)c1 ; CC(C)C(NS(=O)(=O)c1ccc(C(C)(C)C)cc1)C(=O)NO ; Cc1cc(C)c(S(=O)(=O)NC(C(=O)NO)C(C)C)c(C)c1 ; O=C(NO)C1CCCCN1S(=O)(=O)c1ccc(F)cc1 ; O=C(NO)C1CCCCN1S(=O)(=O)c1ccc(C23CC4CC(CC(C4)C2)C3)cc1]

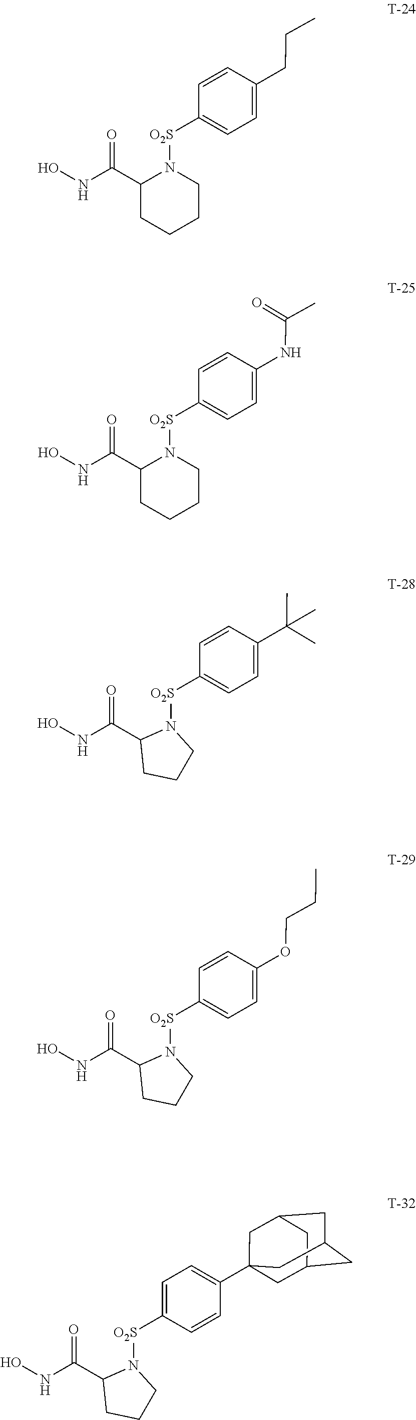 [SMILES: CCCc1ccc(S(=O)(=O)N2CCCCC2C(=O)NO)cc1 ; CC(=O)Nc1ccc(S(=O)(=O)N2CCCCC2C(=O)NO)cc1 ; CC(C)(C)c1ccc(S(=O)(=O)N2CCCC2C(=O)NO)cc1 ; CCCOc1ccc(S(=O)(=O)N2CCCC2C(=O)NO)cc1 ; O=C(NO)C1CCCN1S(=O)(=O)c1ccc(C23CC4CC(CC(C4)C2)C3)cc1]

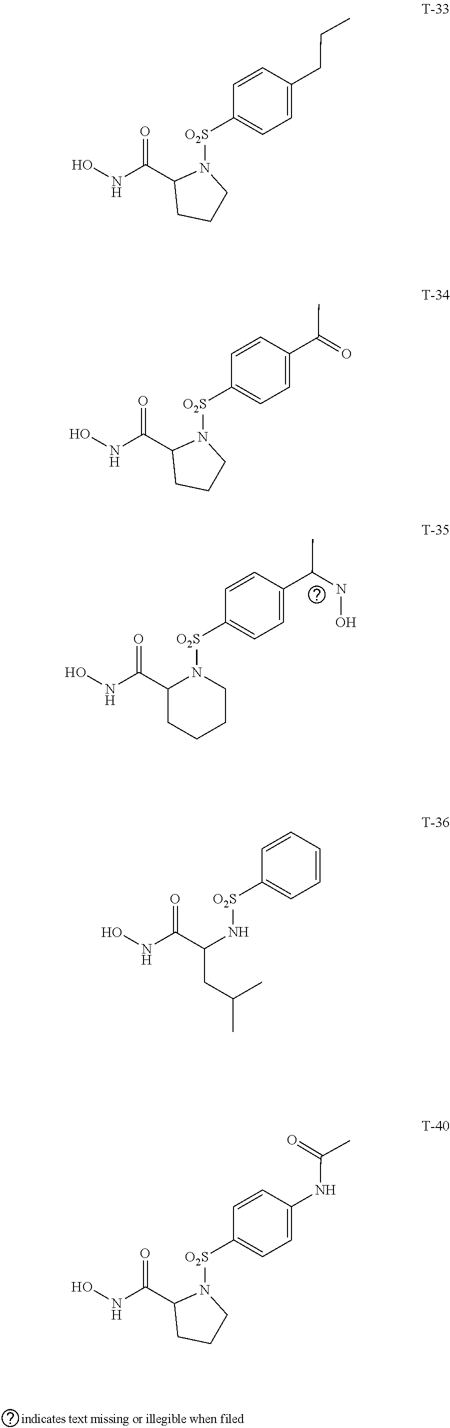 [SMILES: CCCc1ccc(S(=O)(=O)N2CCCC2C(=O)NO)cc1 ; CC(=O)c1ccc(S(=O)(=O)N2CCCC2C(=O)NO)cc1 ; CC(NO)c1ccc(S(=O)(=O)N2CCCCC2C(=O)NO)cc1 ; CC(C)CC(NS(=O)(=O)c1ccccc1)C(=O)NO ; CC(=O)Nc1ccc(S(=O)(=O)N2CCCC2C(=O)NO)cc1]

N-hydroxy-1-(phenylsulfonyl)piperidine-2-carboxamide,
1-([1,1′-biphenyl]-4-ylsulfonyl)-N-hydroxypiperidine-2-carboxamide,
N-hydroxy-2-(naphthalen-2-sulfonamido)-3-phenylpropanamide,
2-([1,1′-biphenyl]-4-sulfonamido)-N-hydroxy-4-methylpentanamide,
2-((4-(tert-butyl)phenyl)sulfonamido)-N-hydroxy-4-methylpentanamide,
2-((4-(tert-butyl)phenyl)sulfonamido)-N-hydroxy-3-methylbutanamide, and
N-hydroxy-1-((4-propylphenyl)sulfonyl)pyrrolidine-2-carboxamide.

In one embodiment, the compound represented by Chemical Formula 1 may be derived from an amino acid.

For example, the compound represented by Chemical Formula 1 may be derived from any one of the following amino acids.

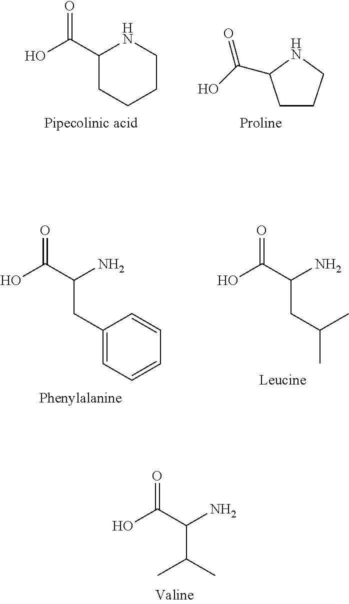

In one embodiment, the composition may be for restoring aged melanin-producing cells to young melanin-producing cells.

In one embodiment, the composition may be for inhibiting a gene expression of one or more of P21 and P16.

In one embodiment, the composition may be for inhibiting melanin production.

In one embodiment, the composition may be for inhibiting mRNA expression of Tyrosinase-related protein 2.

In one embodiment, the composition may be a cosmetic composition.

In one embodiment, the external form of the cosmetic composition may include a dermatologically or cosmetically acceptable medium or base. The composition may be provided in any formulation suitable for topical application, for example, in the form of a solution, gel, solid, paste, anhydrous product, emulsion obtained by dispersing an oil phase in an aqueous phase, suspension, microemulsion, microcapsule, microsphere, ionic (liposomal) or non-ionic vesicular dispersion, or in the form of a cream, toner, lotion, powder, ointment, spray, or concealer stick. These compositions may be manufactured according to conventional methods in the art.

The composition according to the present disclosure may also be used in the form of a foam, or an aerosol composition further comprising a compressed propellant.

The cosmetic composition according to the present disclosure is not particularly limited in its formulation, and, for example, may be formulated into cosmetics such as a softening toner, an astringent toner, a nourishing toner, a nourishing cream, a massage cream, an essence, an eye cream, an eye essence, a cleansing cream, a cleansing foam, a cleansing water, a cleansing tissue including the cosmetic composition described above, a facial pack, a powder, a body lotion, a body cream, a body oil, or a body essence.

When the formulation of the cosmetic composition according to the present disclosure is a paste, cream, or gel, carrier components such as animal fibers, plant fibers, waxes, paraffin, starch, tragacanth, cellulose derivatives, polyethylene glycol, silicone, bentonite, silica, talc, zinc oxide, or the like may be used.

When the formulation of the cosmetic composition according to the present disclosure is a powder or a spray, carrier components such as lactose, talc, silica, aluminum hydroxide, calcium silicate, or polyamide powder may be used, and particularly in the case of a spray, it may further include a propellant such as chlorofluorohydrocarbon, propane/butane, or dimethyl ether.

When the formulation of the cosmetic composition according to the present disclosure is a solution or an emulsion, carrier components such as solvents, solubilizing agents, or emulsifiers may be used, for example, water, ethanol, isopropanol, ethyl carbonate, ethyl acetate, benzyl alcohol, benzyl benzoate, propylene glycol, 1,3-butylene glycol oil, glycerol aliphatic ester, polyethylene glycol, or sorbitan fatty acid ester.

When the formulation of the cosmetic composition according to the present disclosure is a suspension, carrier components such as liquid diluents (e.g., water, ethanol, or propylene glycol), suspending agents such as ethoxylated isostearyl alcohol, polyoxyethylene sorbitol ester, and polyoxyethylene sorbitan ester, or microcrystalline cellulose, aluminum metahydroxide, bentonite, agar, or tragacanth may be used.

The cosmetic composition according to the present disclosure may further include functional additives and ingredients commonly included in general cosmetic compositions. The functional additive may include a component selected from the group consisting of water-soluble vitamins, fat-soluble vitamins, polymer peptides, polymeric polysaccharides, squalane, sphingolipids, and seaweed extracts.

The cosmetic composition according to the present disclosure may also include, in addition to the functional additive, ingredients commonly used in general cosmetic compositions, as needed. Additional blending ingredients may include oil components, moisturizers, emollients, emulsifiers, organic or inorganic pigments, organic powders, ultraviolet absorbers, preservatives, antiseptics, antioxidants, plant extracts, pH adjusters, alcohols, colorants, fragrances, blood circulation promoters, cooling agents, antiperspirants, purified water, and the like.

In one embodiment, the composition may be for skin external application.

The skin external application may include any preparation applied externally to the skin and is a general term encompassing various formulations of cosmetics pharmaceuticals, and the like.

In one embodiment, the composition may be a food composition.

The food composition according to the present disclosure may be in a liquid or solid formulation, and may be in the form of a tablet, a capsule, a soft capsule, a pill, a granule, a beverage (drink type), a diet bar, a chocolate, a caramel formulation, or a confectionery formulation, and its formulation is not particularly limited. The food composition according to the present disclosure may, in addition to the active ingredient described above, appropriately include, as needed, excipients, saccharides, flavoring agents, colorants, oils and fats, proteins, or the like.

In one embodiment, the composition may be a pharmaceutical composition.

The pharmaceutical composition may further include pharmaceutical auxiliaries such as preservatives, stabilizers, hydrating agents, emulsifying promoters, salts and/or buffers, etc. for osmotic pressure control, and other therapeutically useful substances, and may be formulated into various oral or parenteral dosage forms according to conventional methods.

The oral dosage forms may include, for example, tablets, pills, hard and soft capsules, liquid preparations, suspensions, emulsions, syrups, powders, loose powders, fine granules, granules, pellets, and the like, which may contain, in addition to the active ingredient, surfactants, diluents (e.g., lactose, dextrose, sucrose, mannitol, sorbitol, cellulose, and glycine), and lubricants (e.g., silica, talc, stearic acid and its magnesium or calcium salts, and polyethylene glycol). The tablets may further contain binding agents such as magnesium aluminum silicate, starch paste, gelatin, tragacanth, methylcellulose, sodium carboxymethylcellulose, and polyvinylpyrrolidone, and, if necessary, may include disintegrating agents such as starch, agar, alginic acid, or sodium alginate, as well as absorbents, coloring agents, flavoring agents, and sweeteners as pharmaceutical additives. The tablets may be manufactured by conventional mixing, granulation, or coating methods. In addition, the parenteral dosage forms may be transdermal formulations, and may include, for example, injections, drops, ointments, lotions, gels, creams, sprays, suspensions, emulsions, suppositories, patches or the like, but are not limited thereto.

The pharmaceutical composition may be administered parenterally, rectally, topically, transdermally, subcutaneously, or the like.

In one embodiment, the pharmaceutical composition may be for preventing, improving, or treating skin hyperpigmentation.

In one embodiment, the pharmaceutical composition may be for treating a subject in which a gene expression of one or more of P21 or P16 is activated.

In one embodiment, the pharmaceutical composition may be for treating a subject in which melanin production is activated.

In one embodiment, the pharmaceutical composition may be for treating a subject in which mRNA expression of Tyrosinase-related protein 2 is activated.

As used herein, the term “prevention” refers to any act of inhibiting or delaying the onset of a disease by administration of the pharmaceutical composition according to the present disclosure.

As used herein, the term “improvement” refers to any act of reducing, at least, a parameter related to the treated condition, for example, the degree of symptoms.

As used herein, the term “treatment” refers to any act of alleviating or favorably changing symptoms caused by a disease through administration of the pharmaceutical composition according to the present disclosure.

In one embodiment, the content of the active ingredient may be 0.01 μM to 100 mM based on the total volume of the composition.

For example, the content may be 0.01 μM or more, 0.05 μM or more, 0.1 μM or more, 1 μM or more, 10 μM or more, 100 μM or more, 0.5 mM or more, 1 mM or more, 1.5 mM or more, 2 mM or more, 2.5 mM or more, 10 mM or more, or 50 mM or more, and may be 100 mM or less, 50 mM or less, 10 mM or less, 8 mM or less, 6 mM or less, 4 mM or less, 1 mM or less, 100 μM or less, 10 μM or less, 1 μM or less, 0.1 μM or less, or 0.05 μM or less.

For example, the content may be 0.01 μM to 100 mM, 0.05 μM to 10 mM, or 0.1 μM to 4 mM based on the total volume of the composition.

When the concentration is less than 0.01 μM, the effect may be insignificant, and when it is more than 100 mM, there may be cytotoxicity due to high concentration.

In one embodiment, the daily application amount of the active ingredient may be 1 to 1000 mg/kg.

For example, the daily application amount may be a daily administration amount, or may mean a daily intake amount as a human intake amount.

For example, the daily application amount may be 1 mg/kg or more, 10 mg/kg or more, 100 mg/kg or more, 300 mg/kg or more, 500 mg/kg or more, 700 mg/kg or more, or 900 mg/kg or more, and may be 1000 mg/kg or less, 800 mg/kg or less, 600 mg/kg or less, 400 mg/kg or less, 200 mg/kg or less, 100 mg/kg or less, or 10 mg/kg or less.

In one embodiment, the active ingredient may be treated to cells at a concentration of 1 to 100 μM.

For example, the active ingredient may be treated to cells at a concentration of 1 M or more, 10 μM or more, 30 μM or more, 50 μM or more, 70 μM or more, or 90 μM or more, and may be treated to cells at a concentration of 100 μM or less, 80 μM or less, 60 μM or less, 40 μM or less, or 20 μM or less.

Hereinafter, the present disclosure will be described in more detail with reference to the following embodiments. However, the following embodiments are provided for illustrative purposes only to assist in understanding the present disclosure and are not intended to limit the scope and range of the present disclosure.

### Example

### Manufacturing Example

New compounds were manufactured as follows, and IUPAC names of twenty-five new compounds are shown in Table 1 below.

TABLE 1

| TM No. | Compound Name |
|---|---|
| T-01 | N-hydroxy-1-(phenylsulfonyl)piperidine-2-carboxamide |
| T-02 | 1-([1,1′-biphenyl]-4-ylsulfonyl)-N-hydroxypiperidine-2-carboxamide |
| T-04 | 1-((4-butoxyphenyl)sulfonyl)-N-hydroxypiperidine-2-carboxamide |
| T-05 | N-hydroxy-1-(mesitylsulfonyl)piperidine-2-carboxamide |
| T-08 | 2-((4-(tert-butyl)phenyl)sulfonamido)-N-hydroxy-3-phenylpropanamide |
| T-10 | N-hydroxy-3-phenyl-2-((2,4,6- |
| | trimethylphenyl)sulfonamido)propanamide |
| T-11 | N-hydroxy-2-(naphthalen-2-sulfonamido)-3-phenylpropanamide |
| T-12 | 2-([1,1′-biphenyl]-4-sulfonamido)-N-hydroxy-4-methylpentanamide |
| T-13 | 2-((4-(tert-butyl)phenyl)sulfonamido)-N-hydroxy-4-methylpentanamide |
| T-14 | 2-((4-butoxyphenyl)sulfonamido)-N-hydroxy-4-methylpentanamide |
| T-15 | N-hydroxy-4-methyl-2-((2,4,6- |
| | trimethylphenyl)sulfonamido)pentanamide |
| T-18 | 2-((4-(tert-butyl)phenyl)sulfonamido)-N-hydroxy-3-methylbutanamide |
| T-20 | N-hydroxy-3-methyl-2-((2,4,6-trimethylphenyl)sulfonamido)butanamide |
| T-21 | 1-((4-fluorophenyl)sulfonyl)-N-hydroxypiperidine-2-carboxamide |
| T-23 | 1-((4-((3r,5r,7r)-adamantan-1-yl)phenyl)sulfonyl)-N-hydroxypiperidine- |
| | 2-carboxamide |
| T-24 | N-hydroxy-1-((4-propylphenyl)sulfonyl)piperidine-2-carboxamide |
| T-25 | 1-((4-acetamidophenyl)sulfonyl)-N-hydroxypiperidine-2-carboxamide |
| T-28 | 1-((4-(tert-butyl)phenyl)sulfonyl)-N-hydroxypyrrolidine-2-carboxamide |
| T-29 | 1-((4-butoxyphenyl)sulfonyl)-N-hydroxypyrrolidine-2-carboxamide |
| T-32 | 1-((4-((3r,5r,7r)-adamantan-1-yl)phenyl)sulfonyl)-N- |
| | hydroxypyrrolidine-2-carboxamide |
| T-33 | N-hydroxy-1-((4-propylphenyl)sulfonyl)pyrrolidine-2-carboxamide |
| T-34 | 1-((4-acetylphenyl)sulfonyl)-N-hydroxypyrrolidine-2-carboxamide |
| T-35 | N-hydroxy-1-((4-(1-(hydroxyimino)ethyl)phenyl)sulfonyl)piperidine-2- |
| | carboxamide |
| T-36 | N-hydroxy-4-methyl-2-(phenylsulfonamido)pentanamide |
| T-40 | 1-((4-acetamidophenyl)sulfonyl)-N-hydroxypyrrolidine-2-carboxamide |

### (1) T-01 Synthesis of N-hydroxy-1-(phenylsulfonyl)piperidine-2-carboxamide

1.69 g of pipecolinic acid was stirred well together with 30 mL of tetrahydrofuran (THF), and then 30 mL of 1 μM Na₂CO₃ was added. 2 mL of benzenesulfonyl chloride was slowly added dropwise at 0° C., and the mixture was stirred for 1 hour at the same temperature. After washing with 30 mL of diethyl ether, the pH of the aqueous layer was adjusted to pH 3 by using 3M HCl. After extraction with ethyl acetate, the mixture was dried over MgSO₄, and concentrated under reduced pressure to obtain 1.89 g of the intermediate 1-(phenylsulfonyl)piperidine-2-carboxylic acid. The intermediate was used in the next reaction without a separate purification process.

1.89 g of 1-(phenylsulfonyl)piperidine-2-carboxylic acid was dissolved in 15 mL of THF together with 0.85 mL of N-methylmorpholine-N-oxide (NMO), and the mixture was stirred at 0° C. 0.73 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.73 g of hydroxylamine hydrochloride and 1.46 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:2) to obtain 0.72 g of N-hydroxy-1-(phenylsulfonyl)piperidine-2-carboxamide.

¹H-NMR (500 MHz, DMSO-d₆): δ10.62 (s, 1H), 8.79 (s, 1H), 7.78 (m, 2H), 7.66 (m, 1H), 7.57 (m, 2H), 4.34 (m, 1H), 3.63 (m, 1H), 3.47 (m, 1H), 1.78 (m, 1H), 1.55 (m, 1H) 1.45-1.40 (m, 3H), 1.17 (m, 1H)

### (2) T-02 Synthesis of 1-([1,1′-biphenyl]-4-ylsulfonyl)-N-hydroxypiperidine-2-carboxamide

0.65 g of pipecolinic acid was stirred well together with 20 mL of THF, and then 11.5 mL of 1 μM Na₂CO₃ was added. 1.52 g of 4-biphenylsulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with ethyl acetate, the mixture was dried over MgSO₄, and concentrated under reduced pressure to obtain 0.78 g of the intermediate 1-([1,1′-biphenyl]-4-ylsulfonyl)piperidine-2-carboxylic acid. The intermediate was used in the next reaction without a separate purification process.

0.7 g of 1-([1,1′-biphenyl]-4-ylsulfonyl)piperidine-2-carboxylic acid was dissolved in 20 mL of THF together with 0.25 mL of N-methylmorpholine-N-oxide (NMO), and the mixture was stirred at 0° C. 0.21 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.21 g of hydroxylamine hydrochloride and 0.42 mL of triethylamine in 10 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:2) to obtain 0.26 g of 1-([1,1′-biphenyl]-4-ylsulfonyl)-N-hydroxypiperidine-2-carboxamide.

¹H-NMR (500 MHz, DMSO-d₆): δ10.67 (s, 1H), 8.80 (s, 1H), 7.87 (m, 4H), 7.77 (m, 2H), 7.52 (m, 2H), 7.45 (m, 1H), 4.38 (m, 1H), 3.66 (m, 1H), 3.50 (m, 1H), 1.81 (m, 1H), 1.59 (m, 1H), 1.47-1.43 (m, 3H), 1.26-1.15 (m, 1H)

### (3) T-04 Synthesis of 1-((4-butoxyphenyl)sulfonyl)-N-hydroxypiperidine-2-carboxamide

0.65 g of pipecolinic acid was stirred well together with 20 mL of THF, and then 11.5 mL of 1 μM Na₂CO₃ was added. 1.2 mL of 4-butoxybenzene-1-sulfonyl chloride was slowly added dropwise at 0° C., and the mixture was stirred for 1 hour at room temperature. After washing twice with 50 mL of diethyl ether, the pH of the aqueous layer was adjusted to pH 3 using 3 μM HCl. After extraction with 100 mL of ethyl acetate, the mixture was dried over MgSO₄, and concentrated under reduced pressure to obtain 1.17 g of the intermediate 1-((4-butoxyphenyl)sulfonyl)piperidine-2-carboxylic acid. The intermediate was used in the next reaction without a separate purification process.

1.17 g of 1-((4-butoxyphenyl)sulfonyl)piperidine-2-carboxylic acid was dissolved in 30 mL of THF together with 0.42 mL of N-methylmorpholine-N-oxide (NMO), and the mixture was stirred at 0° C. 0.36 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.36 g of hydroxylamine hydrochloride and 0.72 mL of triethylamine in 20 mL of DMF was then added dropwise to the filtrate. The mixture was stirred at room temperature for 30 minutes, and DMF was concentrated under reduced pressure and removed.

After the addition of ethyl acetate and purified water, layer separation was performed, and the aqueous layer was extracted twice more with ethyl acetate. The combined organic layers were concentrated under reduced pressure, and the obtained solid was purified by column chromatography (hexane:ethyl acetate=1:1) to obtain 0.37 g of 1-((4-butoxyphenyl)sulfonyl)-N-hydroxypiperidine-2-carboxamide.

¹H-NMR (500 MHz, DMSO-d₆): δ10.61 (s, 1H), 8.77 (s, 1H), 7.67 (d, 2H, J=8.5 Hz), 7.06 (d, 2H, J=9 Hz), 4.30 (m, 1H), 4.05 (t, 2H, J=7 Hz), 3.59 (m, 1H), 3.44 (m, 1H) 1.77-1.69 (m, 3H), 1.54-1.42 (m, 6H), 1.18 (m, 1H), 0.94 (t, 3H, J=7.5 Hz)

### (4) T-05 Synthesis of N-hydroxy-1-(mesitylsulfonyl)piperidine-2-carboxamide

0.65 g of pipecolinic acid was stirred well together with 20 mL of THF, and then 11.5 mL of 1 μM Na₂CO₃ was added. 1.31 g of 2-mesitylenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with 100 mL of ethyl acetate, the mixture was dried over MgSO₄, and concentrated under reduced pressure to obtain 0.71 g of the intermediate 1-(mesitylsulfonyl)piperidine-2-carboxylic acid. The intermediate was used in the next reaction without a separate purification process.

0.69 g of 1-(mesitylsulfonyl)piperidine-2-carboxylic acid was dissolved in 20 mL of THF together with 0.27 mL of N-methylmorpholine-N-oxide (NMO), and the mixture was stirred at 0° C. 0.23 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.23 g of hydroxylamine hydrochloride and 0.46 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The combined organic layers were concentrated under reduced pressure, and the obtained solid was purified by column chromatography (hexane:ethyl acetate=1:1) to obtain 0.14 g of N-hydroxy-1-(mesitylsulfonyl)piperidine-2-carboxamide.

¹H-NMR (500 MHz, DMSO-d₆): δ10.48 (s, 1H), 8.86 (s, 1H), 7.05 (s, 2H), 3.69 (t, 1H, J=10.5 Hz), 3.32 (s, 3H), 3.43 (s, 3H) 1.79 (m, 2H), 1.65-1.50 (m, 6H), 1.30-1.23 (m, 3H)

### (5) T-08 Synthesis of 2-((4-(tert-butyl)phenyl)sulfonamido)-N-hydroxy-3-phenylpropanamide

0.83 g of phenylalanine was stirred well together with 20 mL of THF, and then 11.5 mL of 1 μM Na₂CO₃ was added. 1.4 g of 4-tert-butylbenzenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 μM HCl. After extraction with 100 mL of ethyl acetate, the mixture was dried over MgSO₄, and concentrated under reduced pressure to obtain 1.31 g of the intermediate ((4-(tert-butyl)phenyl)sulfonyl)phenylalanine. The intermediate was used in the next reaction without a separate purification process.

1.2 g of ((4-(tert-butyl)phenyl)sulfonyl)phenylalanine was dissolved in 20 mL of THF together with 0.4 mL of N-methylmorpholine-N-oxide (NMO), and the mixture was stirred at 0° C. 0.35 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.35 g of hydroxylamine hydrochloride and 0.69 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The combined organic layers were concentrated under reduced pressure, and the obtained solid was purified by column chromatography (hexane:ethyl acetate=1:1) to obtain 0.38 g of 2-((4-(tert-butyl)phenyl)sulfonamido)-N-hydroxy-3-phenylpropanamide.

¹H-NMR (500 MHz, DMSO-d₆): δ10.61 (s, 1H), 8.88 (s, 1H), 8.13 (d, 1H, J=8.5 Hz), 7.40 (m, 4H), 7.12 (m, 3H), 7.00 (m, 2H), 3.71 (m, 1H), 2.76 (m, 1H), 2.55 (m, 1H), 1.26 (s, 9H)

### (6) T-10 Synthesis of N-hydroxy-3-phenyl-2-((2,4,6-trimethylphenyl)sulfonamido)propanamide

0.83 g of phenylalanine was stirred well together with 20 mL of THF, and then 11.5 mL of 1 μM Na₂CO₃ was added. 1.31 g of 2-mesitylenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with 100 mL of ethyl acetate, the mixture was dried over MgSO₄, and concentrated under reduced pressure to obtain 1 g of the intermediate (mesitylsulfonyl)phenylalanine. The intermediate was used in the next reaction without a separate purification process.

1 g of (mesitylsulfonyl)phenylalanine was dissolved in 20 mL of THF together with 0.35 mL of N-methylmorpholine-N-oxide (NMO), and the mixture was stirred at 0° C. 0.3 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.3 g of hydroxylamine hydrochloride and 0.6 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.25 g of N-hydroxy-3-phenyl-2-((2,4,6-trimethylphenyl)sulfonamido)propanamide.

¹H-NMR (500 MHz, DMSO-d₆): δ10.59 (s, 1H), 8.87 (s, 1H), 7.89 (d, 1H, J=9.5 Hz), 7.07 (m, 3H), 6.95 (m, 2H), 6.82 (m, 2H), 3.67 (m, 1H), 2.76 (m, 1H), 2.60 (m, 1H), 2.49 (s, 6H), 2.26 (s, 3H)

### (7) T-11 Synthesis of N-hydroxy-2-(naphthalen-2-sulfonamido)-3-phenylpropanamide

0.83 g of phenylalanine was stirred well together with 20 mL of THF, and then 11.5 mL of 1 μM Na₂CO₃ was added. 1.36 g of 2-naphthalenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with 100 mL of ethyl acetate, the mixture was dried over MgSO₄, and concentrated under reduced pressure to obtain 1.2 g of the intermediate (naphthalen-2-ylsulfonyl)phenylalanine. The intermediate was used in the next reaction without a separate purification process.

1.1 g of (naphthalen-2-ylsulfonyl)phenylalanine was dissolved in 20 mL of THF together with 0.37 mL of N-methylmorpholine-N-oxide (NMO), and the mixture was stirred at 0° C. 0.32 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.32 g of hydroxylamine hydrochloride and 0.65 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:2) to obtain 0.21 g of N-hydroxy-2-(naphthalen-2-sulfonamido)-3-phenylpropanamide.

¹H-NMR (500 MHz, DMSO-d₆): δ10.63 (s, 1H), 8.84 (s, 1H), 8.32 (d, 1H, J=9.5 Hz), 8.19 (s, 1H), 8.19 (m, 2H), 7.92 (d, 1H, J=9.5 Hz), 7.65 (m, 2H), 7.56 (m, 1H), 7.28-7.00 (m, 5H), 3.84 (q, 1H), 2.78 (m, 1H), 2.59 (m, 1H)

### (8) T-12 Synthesis of 2-([1,1′-biphenyl]-4-sulfonamido)-N-hydroxy-4-methylpentanamide

0.66 g of leucine was stirred well together with 20 mL of THF, and then 11.5 mL of 1 M Na₂CO₃ was added. 1.52 g of 4-biphenylsulfonyl chloride was slowly added dropwise at 0° C., and the mixture was stirred for 1 hour at room temperature. After washing twice with 50 mL of diethyl ether, the pH of the aqueous layer was adjusted to pH 3 using 3 μM HCl. After extraction with 100 mL of ethyl acetate, the mixture was dried over MgSO₄, and concentrated under reduced pressure to obtain 0.64 g of the intermediate ([1,1′-biphenyl]-4-ylsulfonyl)leucine. The intermediate was used in the next reaction without a separate purification process.

0.6 g of ([1,1′-biphenyl]-4-ylsulfonyl)leucine was dissolved in 20 mL of THF together with 0.21 mL of N-methylmorpholine-N-oxide (NMO), and the mixture was stirred at 0° C. 0.18 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.18 g of hydroxylamine hydrochloride and 0.36 mL of triethylamine in 10 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.34 g of 2-([1,1′-biphenyl]-4-sulfonamido)-N-hydroxy-4-methylpentanamide.

¹H-NMR (500 MHz, DMSO-d₆): δ10.66 (s, 1H), 8.81 (s, 1H), 8.03 (d, 1H, J=6.0 Hz), 7.83 (m, 4H), 7.73 (d, 2H, J=7.0 Hz), 7.51 (m, 2H), 7.43 (m, 1H), 3.59 (q, 1H), 1.41 (m, 1H), 1.33-1.21 (m, 2H), 0.75 (d, 3H, J=6.5 Hz), 0.64 (d, 3H, J=7.0 Hz)

### (9) T-13 Synthesis of 2-((4-(tert-butyl)phenyl)sulfonamido)-N-hydroxy-4-methylpentanamide

0.66 g of leucine was stirred well together with 20 mL of THF, and then 11.5 mL of 1 M Na₂CO₃ was added. 1.4 g of 4-tert-butylbenzenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with 100 mL of ethyl acetate, the mixture was dried over MgSO₄, and concentrated under reduced pressure to obtain 1.09 g of the intermediate ((4-(tert-butyl)phenyl)sulfonyl)leucine. The intermediate was used in the next reaction without a separate purification process.

1.07 g of ((4-(tert-butyl)phenyl)sulfonyl)leucine was dissolved in 20 mL of THF together with 0.4 mL of N-methylmorpholine-N-oxide (NMO), and the mixture was stirred at 0° C. 0.35 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.35 g of hydroxylamine hydrochloride and 0.7 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.15 g of 2-((4-(tert-butyl)phenyl)sulfonamido)-N-hydroxy-4-methylpentanamide.

¹H-NMR (500 MHz, DMSO-d₆): δ10.64 (s, 1H), 8.83 (s, 1H), 7.88 (m, 1H), 7.70 (d, 2H, J=9.0 Hz), 7.57 (d, 2H, J=8.5 Hz), 3.49 (m, 1H), 1.32-1.24 (m, 11H), 1.12 (m, 1H), 0.69 (d, 3H, J=6.5 Hz), 0.55 (d, 3H, J=6.5 Hz)

### (10) T-14 Synthesis of 2-((4-butoxyphenyl)sulfonamido)-N-hydroxy-4-methylpentanamide

0.66 g of leucine was stirred well together with 20 mL of THF, and then 11.5 mL of 1 M Na₂CO₃ was added. 1.2 mL of 4-butoxybenzene-1-sulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with 100 mL of ethyl acetate, the mixture was dried over MgSO₄, and concentrated under reduced pressure to obtain 1 g of the intermediate ((4-butoxyphenyl)sulfonyl)leucine. The intermediate was used in the next reaction without a separate purification process.

1 g of ((4-butoxyphenyl)sulfonyl)leucine was dissolved in 20 mL of THF together with 0.35 mL of N-methylmorpholine-N-oxide (NMO), and the mixture was stirred at 0° C. 0.3 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.3 g of hydroxylamine hydrochloride and 0.6 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 40 mg of 2-((4-butoxyphenyl)sulfonamido)-N-hydroxy-4-methylpentanamide.

¹H-NMR (500 MHz, DMSO-d₆): δ10.61 (s, 1H), 8.77 (s, 1H), 7.76 (m, 1H), 7.67 (d, 2H, J=9.0 Hz), 7.04 (d, 2H, J=8.5 Hz), 4.04 (t, 2H, J=6.5 Hz), 3.50 (m, 1H), 1.70 (m, 2H), 1.45 (m, 3H), 1.27 (m, 1H), 1.19 (m, 1H), 0.93 (t, 3H, J=7.0 Hz), 0.77-0.72 (d, 3H, J=6.5 Hz), 0.63 (d, 3H, J=6.0 Hz)

### (11) T-15 Synthesis of N-hydroxy-4-methyl-2-((2,4,6-trimethylphenyl)sulfonamido)pentanamide

0.66 g of leucine was stirred well together with 20 mL of THF, and then 11.5 mL of 1 M Na₂CO₃ was added. 1.31 g of 2-mesitylenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 μM HCl.

After extraction with 100 mL of ethyl acetate, the mixture was dried over MgSO₄, and concentrated under reduced pressure to obtain 0.76 g of the intermediate (mesitylsulfonyl)leucine. The intermediate was used in the next reaction without a separate purification process.

0.75 g of (mesitylsulfonyl)leucine was dissolved in 20 mL of THF together with 0.29 mL of N-methylmorpholine-N-oxide (NMO), and the mixture was stirred at 0° C. 0.25 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.25 g of hydroxylamine hydrochloride and 0.5 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.14 g of N-hydroxy-4-methyl-2-((2,4,6-trimethylphenyl)sulfonamido)pentanamide.

¹H-NMR (500 MHz, DMSO-d₆): δ10.51 (s, 1H), 8.88 (s, 1H), 7.77 (m, 1H), 6.98 (s, 2H), 3.45 (m, 1H), 2.55 (s, 3H), 2.50 (s, 3H), 2.24 (s, 3H), 1.41 (m, 1H), 1.33-1.20 (m, 2H), 0.73 (d, 3H, J=6.5 Hz), 0.55 (d, 3H, J=6.5 Hz)

### (12) T-18 Synthesis of 2-((4-(tert-butyl)phenyl)sulfonamido)-N-hydroxy-3-methylbutanamide

0.59 g of valine was stirred well together with 20 mL of THF, and then 11.5 mL of 1 M Na₂CO₃ was added. 1.4 g of 4-tert-butylbenzenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 M HCl. After extraction with 100 mL of ethyl acetate, the mixture was dried over MgSO₄, and concentrated under reduced pressure to obtain 0.71 g of the intermediate ((4-(tert-butyl)phenyl)sulfonyl)valine. The intermediate was used in the next reaction without a separate purification process.

0.7 g of ((4-(tert-butyl)phenyl)sulfonyl)valine was dissolved in 20 mL of THF together with 0.25 mL of N-methylmorpholine-N-oxide (NMO), and the mixture was stirred at 0° C. 0.21 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.21 g of hydroxylamine hydrochloride and 0.42 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.3 g of 2-((4-(tert-butyl)phenyl)sulfonamido)-N-hydroxy-3-methylbutanamide.

¹H-NMR (500 MHz, DMSO-d₆): δ10.50 (s, 1H), 8.80 (s, 1H), 7.81 (m, 1H), 7.68 (d, 2H, J=8.0 Hz), 7.54 (d, 2H, J=8.0 Hz), 3.27 (m, 1H), 1.75 (m, 1H), 1.29 (s, 9H), 0.73 (d, 3H, J=6.5 Hz), 0.70 (d, 3H, J=6.5 Hz)

### (13) T-20 Synthesis of N-hydroxy-3-methyl-2-((2,4,6-trimethylphenyl)sulfonamido)butanamide

0.59 g of valine was stirred well together with 20 mL of THF, and then 11.5 mL of 1 M Na₂CO₃ was added. 1.31 g of 2-mesitylenesulfonyl chloride was slowly added dropwise at 0° C., and the mixture was stirred for 1 hour at room temperature. After washing twice with 50 mL of diethyl ether, the pH of the aqueous layer was adjusted to pH 3 using 3 μM HCl. After extraction with 100 mL of ethyl acetate, the mixture was dried over MgSO₄, and concentrated under reduced pressure to obtain 0.53 g of the intermediate (mesitylsulfonyl)valine. The intermediate was used in the next reaction without a separate purification process.

0.52 g of (mesitylsulfonyl)valine was dissolved in 20 mL of THF together with 0.25 mL of N-methylmorpholine-N-oxide (NMO), and the mixture was stirred at 0° C. 0.21 mL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 0.21 g of hydroxylamine hydrochloride and 0.42 mL of triethylamine in 20 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.14 g of N-hydroxy-3-methyl-2-((2,4,6-trimethylphenyl)sulfonamido)butanamide.

¹H-NMR (500 MHz, DMSO-d₆): δ10.43 (s, 1H), 8.80 (s, 1H), 7.59 (d, 1H, J=9.5 Hz), 6.98 (s, 2H), 3.18 (t, 1H, J=8.0 Hz), 2.59-2.50 (m, 6H), 2.24 (s, 3H), 1.76 (m, 1H), 0.70 (m, 6H)

### (14) T-21 Synthesis of 1-((4-fluorophenyl)sulfonyl)-N-hydroxypiperidine-2-carboxamide

500 mg of pipecolinic acid was stirred well together with 8 mL of THF, and then 9.6 mL of 1 μM Na₂CO₃ was added. 904 mg of 4-fluorobenzenesulfonyl chloride was slowly added dropwise at 0° C., and the mixture was stirred for 2 hours at room temperature. After washing twice with 20 mL of diethyl ether, the pH of the aqueous layer was adjusted to pH 2 using 3 μM HCl. After extraction twice with 20 mL of ethyl acetate, the mixture was dried over MgSO₄, and concentrated under reduced pressure to obtain 608 mg of the intermediate 1-((4-fluorophenyl)sulfonyl)piperidine-2-carboxylic acid. The intermediate was used in the next reaction without a separate purification process.

550 mg of 1-((4-fluorophenyl)sulfonyl)piperidine-2-carboxylic acid was dissolved in 6 mL of THF together with 0.23 mL of N-methylmorpholine-N-oxide (NMO), and the mixture was stirred at 0° C. 0.2 mL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 0.2 g of hydroxylamine hydrochloride and 0.4 mL of triethylamine in 2 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 0.14 g of 1-((4-fluorophenyl)sulfonyl)-N-hydroxypiperidine-2-carboxamide.

¹H-NMR (500 MHz, DMSO-d₆): δ10.63 (s, 1H), 8.76 (s, 1H), 7.82 (m, 2H), 7.42 (m, 2H), 4.32 (m, 1H), 3.62 (m, 1H), 3.46 (m, 1H), 1.80 (m, 1H), 1.77 (m, 1H), 1.51-1.39 (m, 3H), 1.19 (m, 1H)

### (15) T-23 Synthesis of 1-((4-((3r,5r,7r)-adamantan-1-yl)phenyl)sulfonyl)-N-hydroxypiperidine-2-carboxamide

50 mg of pipecolinic acid was stirred well together with 0.8 mL of THF, and then 0.9 mL of 1 μM Na₂CO₃ was added. 144 mg of 4-(1-adamantyl)benzenesulfonyl chloride was slowly added dropwise at 0° C., and the mixture was stirred for 2 hours at room temperature. After washing twice with 2 mL of diethyl ether, the pH of the aqueous layer was adjusted to pH 2 using 3 μM HCl. After extraction twice with 2 mL of ethyl acetate, the mixture was dried over MgSO₄, and concentrated under reduced pressure to obtain 90 mg of the intermediate 1-((4-((3r,5r,7r)-adamantan-1-yl)phenyl)sulfonyl)piperidine-2-carboxylic acid. The intermediate was used in the next reaction without a separate purification process.

50 mg of 1-((4-((3r,5r,7r)-adamantan-1-yl)phenyl)sulfonyl)piperidine-2-carboxylic acid was dissolved in 1 mL of THF together with 15 μL of N-methylmorpholine-N-oxide (NMO), and the mixture was stirred at 0° C. 13 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 12.9 mg of hydroxylamine hydrochloride and 26 μL of triethylamine in 1 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 30 mg of 1-((4-((3r,5r,7r)-adamantan-1-yl)phenyl)sulfonyl)-N-hydroxypiperidine-2-carboxamide.

¹H-NMR (500 MHz, DMSO-d₆): δ10.61 (s, 1H), 8.81 (s, 1H), 7.70 (m, 2H), 7.55 (m, 2H), 4.33 (m, 1H), 3.59 (m, 1H), 3.45 (m, 1H), 2.07 (s, 3H), 1.89 (m, 7H), 1.77 (m, 7H), 1.52 (m, 1H), 1.43 (m, 2H), 1.19 (m, 1H)

### (16) T-24 Synthesis of N-hydroxy-1-((4-propylphenyl)sulfonyl)piperidine-2-carboxamide

500 mg of pipecolinic acid was stirred well together with 8 mL of THF, and then 8.9 mL of 1 μM Na₂CO₃ was added. 831 μL of 4-n-propylbenzenesulfonyl chloride was slowly added dropwise at 0° C., and the mixture was stirred for 2 hours at room temperature. After washing twice with 10 mL of diethyl ether, the pH of the aqueous layer was adjusted to pH 2 using 3 μM HCl. After extraction twice with 20 mL of ethyl acetate, the mixture was dried over MgSO₄, and concentrated under reduced pressure to obtain 888 mg of the intermediate 1-((4-propylphenyl)sulfonyl)piperidine-2-carboxylic acid. The intermediate was used in the next reaction without a separate purification process.

700 mg of 1-((4-propylphenyl)sulfonyl)piperidine-2-carboxylic acid was dissolved in 6 mL of THF together with 272 μL of N-methylmorpholine-N-oxide (NMO), and the mixture was stirred at 0° C. 236 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 234 mg of hydroxylamine hydrochloride and 470 μL of triethylamine in 4 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 0.16 g of N-hydroxy-1-((4-propylphenyl)sulfonyl)piperidine-2-carboxamide.

¹H-NMR (500 MHz, DMSO-d₆): δ10.61 (s, 1H), 8.79 (s, 1H), 7.65 (m, 2H), 7.38 (m, 2H), 4.33 (m, 1H), 3.61 (m, 1H), 3.45 (m, 1H), 2.43 (t, 2H, J=7.5 Hz), 1.76 (m, 1H), 1.59-1.38 (m, 6H), 1.16 (m, 1H), 0.90 (t, 3H, J=7.5 Hz)

### (17) T-25 Synthesis of 1-((4-acetamidophenyl)sulfonyl)-N-hydroxypiperidine-2-carboxamide

500 mg of pipecolinic acid was stirred well together with 8 mL of THF, and then 8.9 mL of 1 μM Na₂CO₃ was added. 1.09 g of N-acetylsulfanilyl chloride was slowly added dropwise at 0° C., and the mixture was stirred for 2 hours at room temperature. After washing twice with 10 mL of diethyl ether, the pH of the aqueous layer was adjusted to pH 2 using 3 M HCl. After extraction twice with 20 mL of ethyl acetate, the mixture was dried over MgSO₄, and concentrated under reduced pressure to obtain 935 mg of the intermediate 1-((4-acetamidophenyl)sulfonyl)piperidine-2-carboxylic acid. The intermediate was used in the next reaction without a separate purification process.

800 mg of 1-((4-acetamidophenyl)sulfonyl)piperidine-2-carboxylic acid was dissolved in 6 mL of THF together with 296 μL of N-methylmorpholine-N-oxide (NMO), and the mixture was stirred at 0° C. 258 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 256 mg of hydroxylamine hydrochloride and 512 μL of triethylamine in 4 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 0.18 g of 1-((4-acetamidophenyl)sulfonyl)-N-hydroxypiperidine-2-carboxamide.

¹H-NMR (500 MHz, DMSO-d₆): δ10.60 (s, 1H), 10.32 (s, 1H), 8.78 (s, 1H), 7.74 (m, 2H), 7.68 (m, 2H), 4.30 (m, 1H), 3.61 (m, 1H), 3.45 (m, 1H), 2.09 (s, 3H), 1.76 (m, 1H), 1.51 (m, 1H), 1.39 (m, 3H), 1.16 (m, 1H)

### (18) T-28 Synthesis of 1-((4-(tert-butyl)phenyl)sulfonyl)-N-hydroxypyrrolidine-2-carboxamide

500 mg of proline was stirred well together with 9 mL of THF, and then 10 mL of 1 M Na₂CO₃ was added. 1.21 g of 4-tert-butylbenzenesulfonyl chloride was slowly added dropwise at 0° C., and the mixture was stirred for 2 hours at room temperature. After washing twice with 10 mL of diethyl ether, the pH of the aqueous layer was adjusted to pH 2 using 3 M HCl. After extraction twice with 20 mL of ethyl acetate, the mixture was dried over MgSO₄, and concentrated under reduced pressure to obtain 860 mg of the intermediate ((4-(tert-butyl)phenyl)sulfonyl)proline. The intermediate was used in the next reaction without a separate purification process.

700 mg of ((4-(tert-butyl)phenyl)sulfonyl)proline was dissolved in 5 mL of THF together with 272 μL of N-methylmorpholine-N-oxide (NMO), and the mixture was stirred at 0° C. 236 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 234 mg of hydroxylamine hydrochloride and 470 μL of triethylamine in 4 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 546 mg of 1-((4-(tert-butyl)phenyl)sulfonyl)-N-hydroxypyrrolidine-2-carboxamide.

¹H-NMR (500 MHz, DMSO-d₆): δ10.66 (s, 1H), 8.94 (s, 1H), 7.78 (m, 2H), 7.64 (m, 2H), 3.91 (m, 1H), 3.30 (m, 1H), 3.13 (m, 1H), 1.88 (m, 1H) 1.75 (m, 1H) 1.66 (m, 1H), 1.46 (m, 1H), 1.31 (s, 9H)

### (19) T-29 Synthesis of 1-((4-butoxyphenyl)sulfonyl)-N-hydroxypyrrolidine-2-carboxamide

500 mg of proline was stirred well together with 9 mL of THF, and then 10 mL of 1 M Na₂CO₃ was added. 1.30 g of 4-butoxybenzenesulfonyl chloride was slowly added dropwise at 0° C., and the mixture was stirred for 2 hours at room temperature. After washing twice with 10 mL of diethyl ether, the pH of the aqueous layer was adjusted to pH 2 using 3 μM HCl. After extraction twice with 20 mL of ethyl acetate, the mixture was dried over MgSO₄, and concentrated under reduced pressure to obtain 1.2 g of the intermediate ((4-butoxyphenyl)sulfonyl)proline. The intermediate was used in the next reaction without a separate purification process.

1 g of ((4-butoxyphenyl)sulfonyl)proline was dissolved in 7 mL of THF together with 369 μL of N-methylmorpholine-N-oxide (NMO), and the mixture was stirred at 0° C. 321 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 318 mg of hydroxylamine hydrochloride and 639 μL of triethylamine in 4 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 370 mg of 1-((4-butoxyphenyl)sulfonyl)-N-hydroxypyrrolidine-2-carboxamide.

¹H-NMR (500 MHz, DMSO-d₆): δ10.65 (s, 1H), 8.93 (s, 1H), 7.76 (m, 2H), 7.12 (m, 2H), 4.07 (t, 2H, J=6.5 Hz), 3.88 (m, 1H), 3.39 (m, 1H), 3.11 (m, 1H), 1.83 (m, 1H), 1.74 (m, 3H), 1.63 (m, 1H), 1.45 (m, 3H), 0.94 (t, 3H, J=8.5 Hz)

### (20) T-32 Synthesis of 1-((4-((3r,5r,7r)-adamantan-1-yl)phenyl)sulfonyl)-N-hydroxypyrrolidine-2-carboxamide

50 mg of proline was stirred well together with 0.9 mL of THF, and then 1 mL of 1 M Na₂CO₃ was added. 162 mg of 4-(1-adamantyl)benzenesulfonyl chloride was slowly added dropwise at 0° C., and the mixture was stirred for 2 hours at room temperature. After washing twice with 2 mL of diethyl ether, the pH of the aqueous layer was adjusted to pH 2 using 3 M HCl. After extraction twice with 2 mL of ethyl acetate, the mixture was dried over MgSO₄, and concentrated under reduced pressure to obtain 171 mg of the intermediate ((4-((3r,5r,7r)-adamantan-1-yl)phenyl)sulfonyl)proline. The intermediate was used in the next reaction without a separate purification process.

130 mg of ((4-((3r,5r,7r)-adamantan-1-yl)phenyl)sulfonyl)proline was dissolved in 2 mL of THF together with 40 μL of N-methylmorpholine-N-oxide (NMO), and the mixture was stirred at 0° C. 35 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 35 mg of hydroxylamine hydrochloride and 70 μL of triethylamine in 1 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 90 mg of 1-((4-((3r,5r,7r)-adamantan-1-yl)phenyl)sulfonyl)-N-hydroxypyrrolidine-2-carboxamide.

¹H-NMR (500 MHz, DMSO-d₆): δ10.66 (s, 1H), 8.95 (s, 1H), 7.77 (m, 2H), 7.62 (m, 2H), 3.91 (m, 1H), 3.39 (m, 1H), 3.12 (m, 1H), 2.15 (s, 3H), 1.89-1.75 (m, 13H), 1.65 (m, 1H), 1.50 (m, 1H), 1.16 (m, 1H)

### (21) T-33 Synthesis of N-hydroxy-1-((4-propylphenyl)sulfonyl)pyrrolidine-2-carboxamide

500 mg of proline was stirred well together with 9 mL of THF, and then 10 mL of 1 M Na₂CO₃ was added. 932 μL of 4-n-propylbenzenesulfonyl chloride was slowly added dropwise at 0° C., and the mixture was stirred for 2 hours at room temperature. After washing twice with 10 mL of diethyl ether, the pH of the aqueous layer was adjusted to pH 2 using 3 M HCl. After extraction twice with 20 mL of ethyl acetate, the mixture was dried over MgSO₄, and concentrated under reduced pressure to obtain 1 g of the intermediate ((4-propylphenyl)sulfonyl)proline. The intermediate was used in the next reaction without a separate purification process.

900 mg of ((4-propylphenyl)sulfonyl)proline was dissolved in 6 mL of THF together with 366 μL of N-methylmorpholine-N-oxide (NMO), and the mixture was stirred at 0° C. 318 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 315 mg of hydroxylamine hydrochloride and 633 μL of triethylamine in 4 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 665 mg of N-hydroxy-1-((4-propylphenyl)sulfonyl)pyrrolidine-2-carboxamide.

¹H-NMR (500 MHz, DMSO-d₆): δ10.66 (s, 1H), 8.94 (s, 1H), 7.76 (m, 2H), 7.46 (m, 2H), 3.90 (m, 1H), 3.38 (m, 1H), 3.14 (m, 1H), 2.66 (t, 2H, J=9 Hz), 1.83 (m, 1H), 1.75 (m, 1H), 1.65 (m, 2H), 1.44 (m, 2H), 0.89 (t, 3H, J=7.5 Hz)

### (22) T-34 Synthesis of 1-((4-acetylphenyl)sulfonyl)-N-hydroxy-pyrrolidine-2-carboxamide

500 mg of proline was stirred well together with 9 mL of THF, and then 10 mL of 1 M Na₂CO₃ was added. 1.14 g of 4-acetylbenzenesulfonyl chloride was slowly added dropwise at 0° C., and the mixture was stirred for 2 hours at room temperature. After washing twice with 10 mL of diethyl ether, the pH of the aqueous layer was adjusted to pH 2 using 3 μM HCl. After extraction twice with 20 mL of ethyl acetate, the mixture was dried over MgSO₄, and concentrated under reduced pressure to obtain 958 mg of the intermediate ((4-acetylphenyl)sulfonyl)proline. The intermediate was used in the next reaction without a separate purification process.

800 mg of ((4-acetylphenyl)sulfonyl)proline was dissolved in 6 mL of THF together with 325 μL of N-methylmorpholine-N-oxide (NMO), and the mixture was stirred at 0° C. 283 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 280 mg of hydroxylamine hydrochloride and 563 μL of triethylamine in 4 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 110 mg of 1-((4-acetylphenyl)sulfonyl)-N-hydroxypyrrolidine-2-carboxamide.

¹H-NMR (500 MHz, DMSO-d₆): δ10.72 (s, 1H), 8.97 (s, 1H), 8.15 (m, 2H), 7.97 (m, 2H), 3.95 (m, 1H), 3.43 (m, 1H), 3.13 (m, 1H), 2.65 (s, 3H), 1.86 (m, 1H), 1.72 (m, 2H), 1.48 (m, 1H)

### (23) T-35 Synthesis of N-hydroxy-1-((4-(1-(hydroxyimino)ethyl)phenyl)sulfonyl)piperidine-2-carboxamide

1 g of pipecolinic acid was stirred well together with 16 mL of THF, and then 17.8 mL of 1 μM Na₂CO₃ was added. 2.04 g of 4-acetylbenzenesulfonyl chloride was slowly added dropwise at 0° C., and the mixture was stirred for 2 hours at room temperature. After washing twice with 20 mL of diethyl ether, the pH of the aqueous layer was adjusted to pH 2 using 3 M HCl. After extraction twice with 40 mL of ethyl acetate, the mixture was dried over MgSO₄, and concentrated under reduced pressure to obtain 1.21 g of the intermediate 1-((4-(1-(hydroxyamino)ethyl)phenyl)sulfonyl)piperidine-2-carboxylic acid. The intermediate was used in the next reaction without a separate purification process.

1 g of 1-((4-(1-(hydroxyamino)ethyl)phenyl)sulfonyl)piperidine-2-carboxylic acid was dissolved in 10 mL of THF together with 388 μL of N-methylmorpholine-N-oxide (NMO), and the mixture was stirred at 0° C. 338 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 446 mg of hydroxylamine hydrochloride and 895 μL of triethylamine in 6 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 0.18 g of N-hydroxy-1-((4-(1-(hydroxyimino)ethyl)phenyl)sulfonyl)piperidine-2-carboxamide.

¹H-NMR (500 MHz, DMSO-d₆): δ11.55 (s, 1H), 10.63 (s, 1H), 8.79 (s, 1H), 7.82 (m, 2H), 7.75 (m, 2H), 4.35 (m, 1H), 3.63 (m, 1H), 3.47 (m, 1H), 3.32 (s, 3H), 1.74 (m, 1H), 1.54 (m, 1H), 1.45 (m, 3H), 1.20 (m, 1H)

### (24) T-36 Synthesis of N-hydroxy-4-methyl-2-(phenylsulfonamido)pentanamide

500 mg of leucine was stirred well together with 8 mL of THF, and then 8.8 mL of 1 M Na₂CO₃ was added. 584 μL of benzenesulfonyl chloride was slowly added dropwise at 0° C., and the mixture was stirred for 1 hour at room temperature. After washing twice with 10 mL of diethyl ether, the pH of the aqueous layer was adjusted to pH 3 using 3 μM HCl. After extraction with 20 mL of ethyl acetate, the mixture was dried over MgSO₄, and concentrated under reduced pressure to obtain 444 mg of the intermediate (phenylsulfonyl)leucine. The intermediate was used in the next reaction without a separate purification process.

400 mg of (phenylsulfonyl)leucine was dissolved in 5 mL of THF together with 178 L of N-methylmorpholine-N-oxide (NMO), and the mixture was stirred at 0° C. 155 μL of ethyl chloroformate was slowly added dropwise, and the reaction mixture was stirred for 30 minutes at the same temperature. After filtration of the solid formed in the reaction mixture, the obtained filtrate was kept separately. A solution prepared by dissolving 154 mg of hydroxylamine hydrochloride and 308 μL of triethylamine in 2 mL of DMF was then added dropwise to the filtrate. The mixture was stirred at room temperature for 30 minutes, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (hexane:ethyl acetate=1:1) to obtain 0.11 g of N-hydroxy-4-methyl-2-(phenylsulfonamido)pentanamide.

¹H-NMR (500 MHz, DMSO-d₆): δ10.65 (s, 1H), 8.81 (s, 1H), 8.00 (m, 1H), 7.77 (m, 2H), 7.57 (m, 1H), 7.55 (m, 2H), 3.48 (m, 1H), 1.43 (m, 1H), 1.35-1.20 (m, 2H), 0.72 (d, 3H, J=6.5 Hz), 0.60 (d, 3H, J=7.0 Hz)

### (25) T-40 Synthesis of 1-((4-acetamidophenyl)sulfonyl)-N-hydroxy-pyrrolidine-2-carboxamide

1 g of proline was stirred well together with 18 mL of THF, and then 20 mL of 1 M Na₂CO₃ was added. 2.44 g of N-acetylsulfanilyl chloride was slowly added dropwise at 0° C., and the mixture was stirred for 2 hours at room temperature. After washing twice with 20 mL of diethyl ether, the pH of the aqueous layer was adjusted to pH 2 using 3 μM HCl. After extraction twice with 40 mL of ethyl acetate, the mixture was dried over MgSO₄, and concentrated under reduced pressure to obtain 483 mg of the intermediate ((4-acetamidophenyl)sulfonyl)proline. The intermediate was used in the next reaction without a separate purification process.

450 mg of ((4-acetamidophenyl)sulfonyl)proline was dissolved in 6 mL of THF together with 174 μL of N-methylmorpholine-N-oxide (NMO), and the mixture was stirred at 0° C. 152 μL of ethyl chloroformate was slowly added dropwise, and the reaction mixture was stirred for 2 hours at the same temperature. After filtration of the solid formed in the reaction mixture, the obtained filtrate was kept separately. A solution prepared by dissolving 200 mg of hydroxylamine hydrochloride and 401 μL of triethylamine in 1.5 mL of DMF was then added dropwise to the filtrate. The mixture was stirred at room temperature for 2 hours, and DMF was concentrated under reduced pressure and removed. After the addition of ethyl acetate and purified water, layer separation was performed, and the aqueous layer was extracted twice more with ethyl acetate. The organic layers were collected and then concentrated under reduced pressure, and the obtained solid was purified by column chromatography (5% methanol (MeOH) in dichloromethane (DCM)) to obtain 120 mg of 1-((4-acetamidophenyl)sulfonyl)-N-hydroxypyrrolidine-2-carboxamide.

¹H-NMR (500 MHz, DMSO-d₆): δ10.64 (s, 1H), 10.38 (s, 1H), 8.91 (s, 1H), 7.79 (m, 4H), 3.90 (m, 1H), 3.41 (m, 1H), 3.11 (m, 1H), 2.09 (s, 3H), 1.82 (m, 1H), 1.70 (m, 2H), 1.42 (m, 1H)

### Experimental Example

The skin anti-aging and skin brightening effects of the new compounds were evaluated through the experiments as follows. Specifically, skin melanin-producing cells that had been aged over time after exposure to low-dose ultraviolet (UV) irradiation were confirmed to exhibit aging phenotypes such as excessive melanin production, enlarged cell size, and the like.

These were developed as aged skin model cells. The active ingredients according to the present disclosure were treated to the cells to confirm anti-aging (reverse-aging) and brightening effects.

### Experimental Example 1—Anti-Aging or Reverse-Aging Effect on Skin

[Preparation of Aged, Hyperpigmented Normal Human Melanin-Producing Model Cells and Treatment with the Test Substance]

Melanin-producing cells (Normal human epidermal melanocytes), isolated from human normal skin, were seeded into a 6-well cell culture plate at a density of 2×10⁴ cells per well. On the following day, after confirming that the cells had adhered and were growing well in the incubator, UVB (312 nm) at 11 mJ was irradiated once every two days, three times in total. After completion of UV irradiation, the cells were cultured for more than one week to prepare an aged, hyperpigmented skin cell model (compound non-treated). The formation of the model cells was confirmed through changes in cell morphology and gene expression. Thereafter, the test compound, 1-([1,1′-biphenyl]-4-ylsulfonyl)-N-hydroxypiperidine-2-carboxamide (T-02), which is a new amide-based compound, was dissolved in DMSO at a 1000× concentration and added to the culture medium to make a final concentration of 10 μM, followed by treatment to the cells. Cells to which only DMSO was added were used as the control group (compound non-treated). The cells were cultured in a 37° C., 5% CO₂ incubator, with the culture medium containing the compound being freshly replaced once every two days for up to four days.

[Evaluation of Reverse-aging Effect from Aged Hyperpigmented Model Cells to Young Cells]

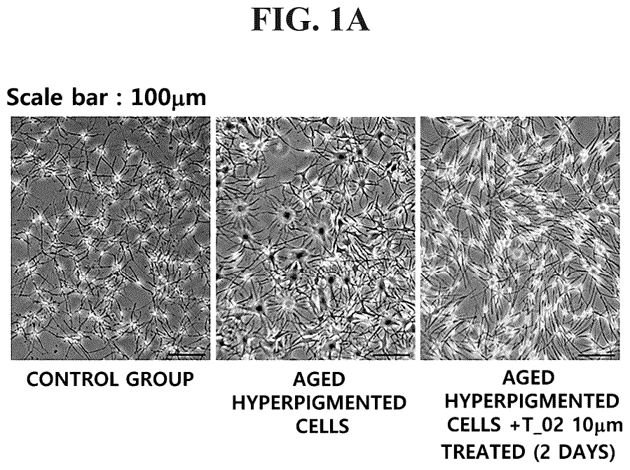

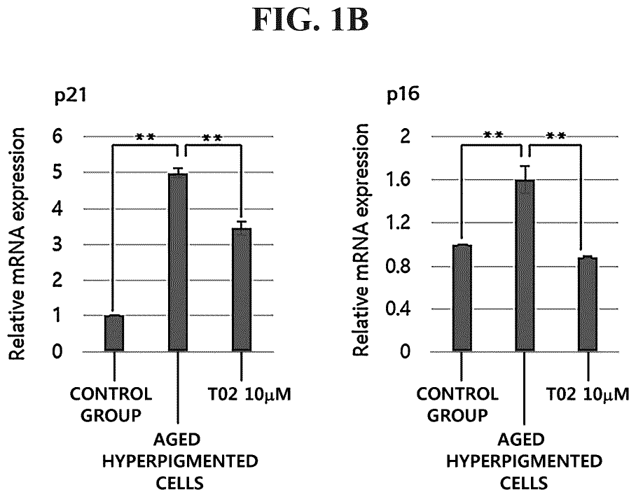

When the amide-based new compound 1-([1,1′-biphenyl]-4-ylsulfonyl)-N-hydroxypiperidine-2-carboxamide (T-02) was treated at a concentration of 10 μM to hyperpigmented model cells that had been excessively aged by repeated exposure to low-dose UVB, it was confirmed that the cell morphology changed from that of aged cells to that of young cells (FIG. 1A).

It is generally known that the increase in cell size upon aging is a common phenotype observed in most types of cells. In addition, in melanin-producing cells in the skin, when melanin is produced and dispersed to surrounding keratin-producing cells (or keratinocytes), the dendrites of melanin-producing cells are involved in this process, and it has been reported that when the number of dendrites increases, melanin dispersion is enhanced, thereby accelerating skin pigmentation.

However, upon treatment with 1-([1,1′-biphenyl]-4-ylsulfonyl)-N-hydroxypiperidine-2-carboxamide (T-02), it was confirmed that the increased cell size due to aging returned to the level of young cells, and the number of cell dendrites decreased and became thinner.

In this case, the RNA expression levels of p21 and p16, which are known as aging-related genes, were both increased in the light-induced accelerated-aging hyperpigmented model cells, whereas treatment with 1-([1,1′-biphenyl]-4-ylsulfonyl)-N-hydroxypiperidine-2-carboxamide (T-02) significantly reduced the elevated expression levels of p21 and p16 in a statistically significant manner (FIG. 1).
(** P<0.01 vs Control group)

### Experimental Example 2—Anti-Aging Effect on Skin

### [Melanin Quantification and Gene Expression Analysis of Aged Hyperpigmented Model Cells]

After completion of the experiment in Experimental Example 1, the cells were photographed using an optical microscope, and the changes in cell size and morphology were observed. After microscopic observation, the cells were collected to an equal number of 2×10⁵ cells per sample, dissolved in 1 N NaOH, and the absorbance was measured at 475 nm to compare the relative amount of melanin. In addition, the cells were lysed in TRIzol and collected, total RNA was extracted, cDNA was synthesized therefrom, and real-time PCR was performed to compare the relative expression levels of each gene. The RNA amount was quantified so that the same amount of RNA was used among experimental groups, and the expression of each gene was normalized to that of the house keeping gene, GAPDH, followed by relative comparison of gene expression levels.

* The gene primers used were purchased from APPLIED BIOSYSTEMS.

Tyrosinase-related protein 2 (Hs01095856_m1), GAPDH (Hs02786624_g1), p21 (Hs00355782_m1), p16 (Hs00923894_m1)

### [Evaluation of the Inhibitory Effect on Melanin Production in Aged Hyperpigmented Model Cells]

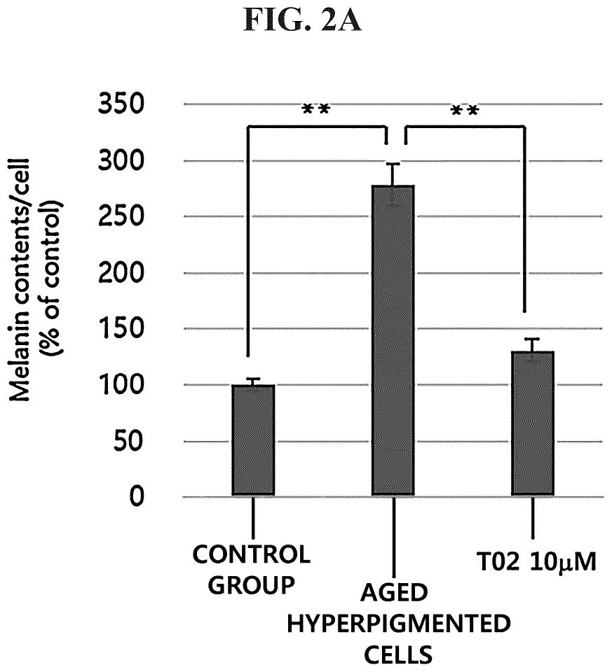

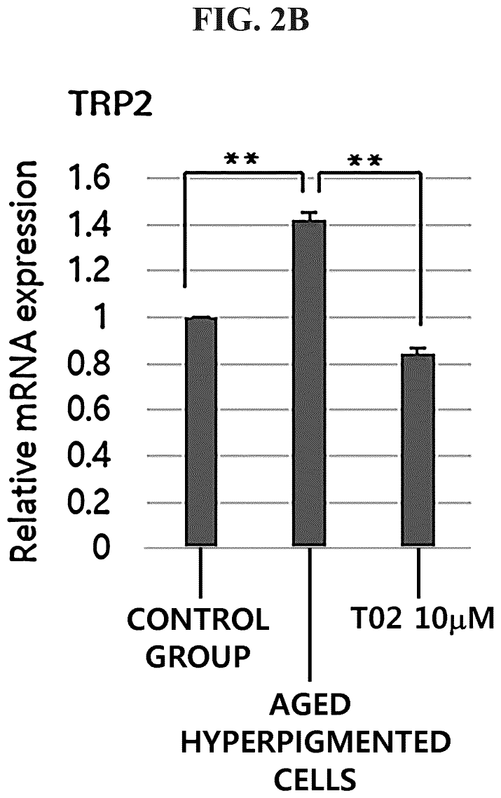

In the case of accelerated-aging hyperpigmented model cells generated by repeated exposure to low-dose UVB, the amount of melanin production per cell increased by almost threefold compared with the control group. However, when 1-([1,1′-biphenyl]-4-ylsulfonyl)-N-hydroxypiperidine-2-carboxamide (T-02) was treated with, the amount of melanin production was confirmed to decrease to almost the level of the control group (FIG. 2A). This observation reflects the aspect in which the melanin that had been generated was decomposed and subsequently removed.

The mRNA expression of Tyrosinase-related protein 2, which is one of the enzymes involved in melanin production, was also increased in the accelerated-aging hyperpigmented model cells. However, it was confirmed that 1-([1,1′-biphenyl]-4-ylsulfonyl)-N-hydroxypiperidine-2-carboxamide (T-02) statistically and significantly inhibited this increased expression (FIG. 2B). This result reflects the aspect in which melanin production was inhibited in advance.
(** P<0.01 vs Control group)

### Experimental Example 3—Brightening Efficacy

The inhibition of gene expression of the melanin-producing enzyme (Tyrosinase-related protein 2, TRP2) and the melanin decomposition effect were observed in the same manner as in Experimental Example 2.

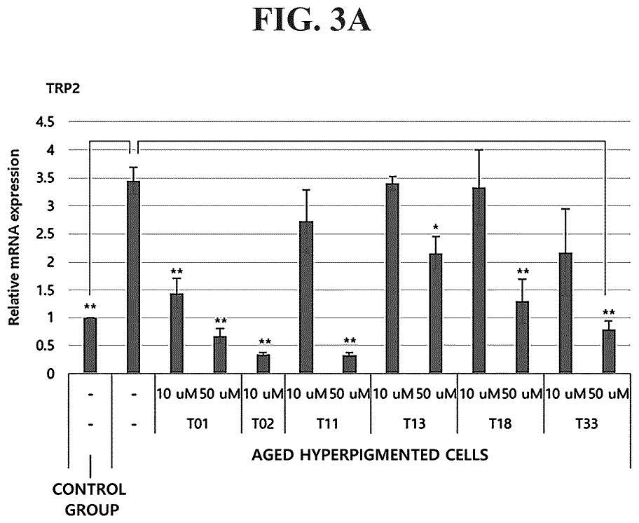

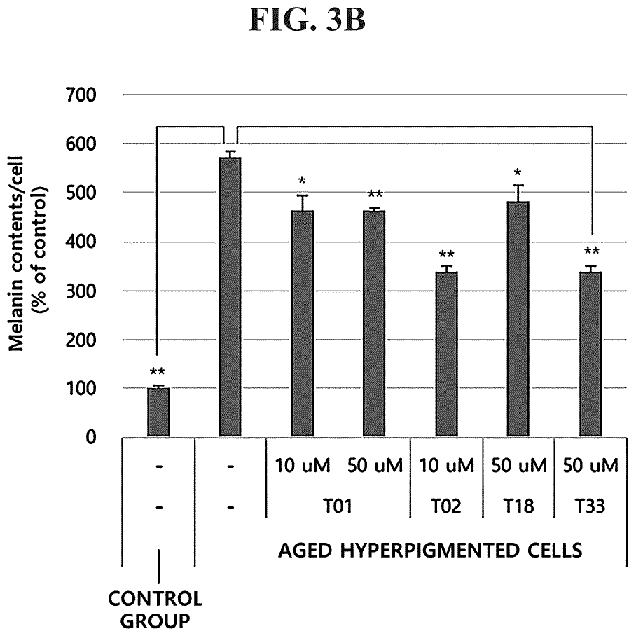

With reference to FIGS. 3A and 3B, it was confirmed that the expression of the TRP2 gene was statistically and significantly inhibited and that the amount of melanin was reduced in T01, T02, T1, T13, T18, and T33.

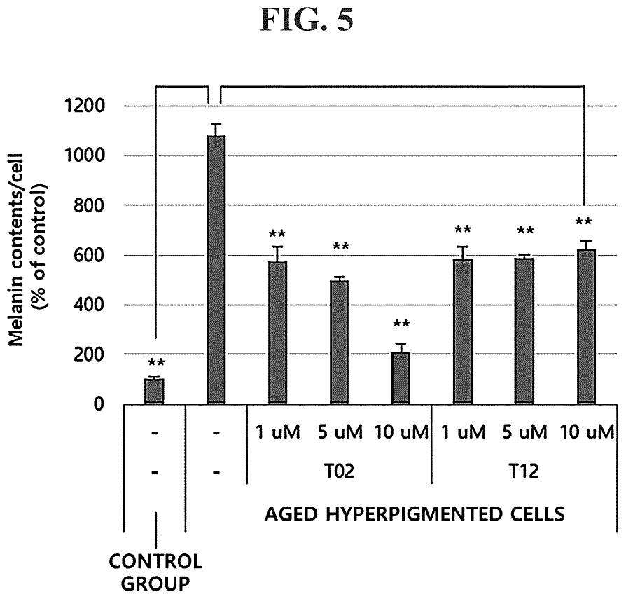

Furthermore, with reference to FIG. 5, it was confirmed that T02 and T12 exhibited excellent melanin-decomposing efficacy.

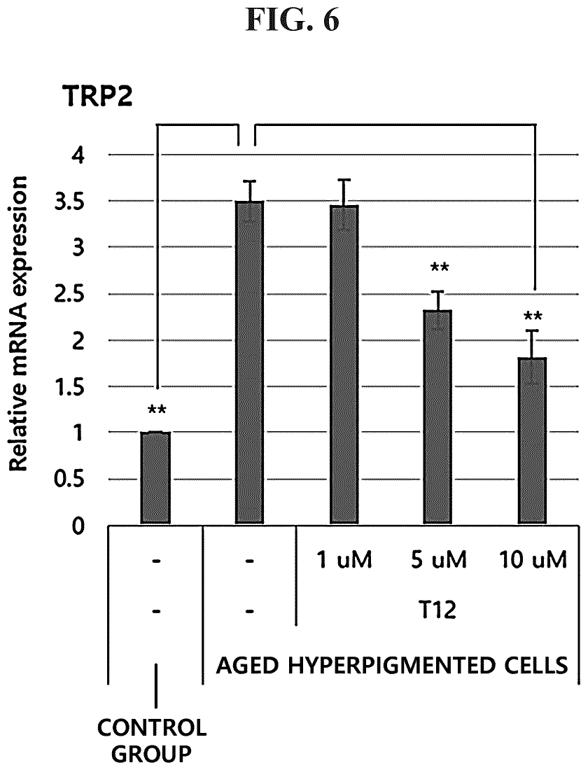

In addition, with reference to FIG. 6, the observation of the inhibition of Tyrosinase-related protein 2 (TRP2) gene expression confirmed that the TRP2 inhibitory efficacy of compound T12 was superior.

In the case of brightening, the inhibition of TRP2 expression may be interpreted as preventive inhibition of new melanin production (anti-aging). However, since the target compound was post-treated on accelerated-aging hyperpigmented model cells by repeated exposure to low-dose UVB, the effect may also be regarded as more of reverse-aging (therapeutic). The melanin-decomposing efficacy can be explained as a therapeutic (reverse-aging) effect resulting from removal or decomposition, since the test material was post-treated on cells in which melanin production had already been excessively induced.

### Experimental Example 4—Anti-Aging or Reverse-Aging Efficacy

The inhibition of RNA expression of the aging-related genes p21 and p16 was observed in the same manner as in Experimental Example 1.

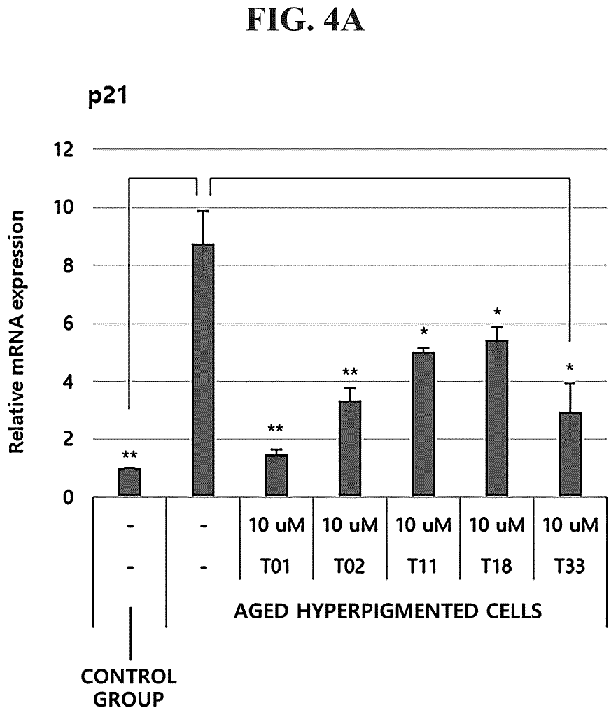

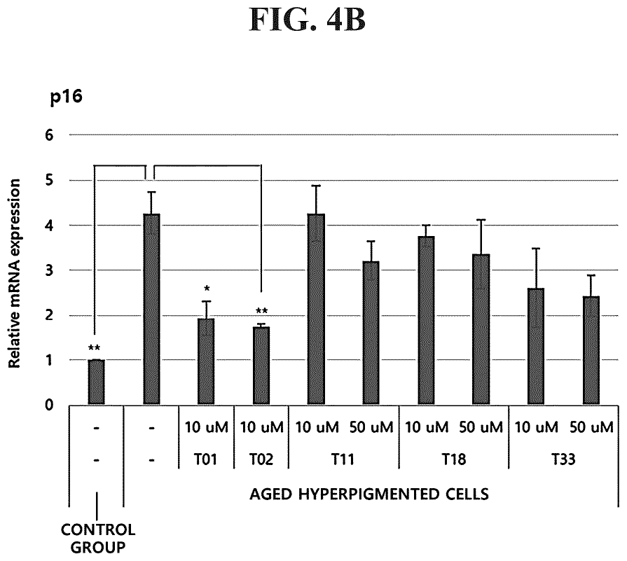

With reference to FIGS. 4A and 4B, it was confirmed that T01, T02, T11, T18, and T33 exhibited statistically and significantly superior inhibitory effects on the expression of aging-related genes.

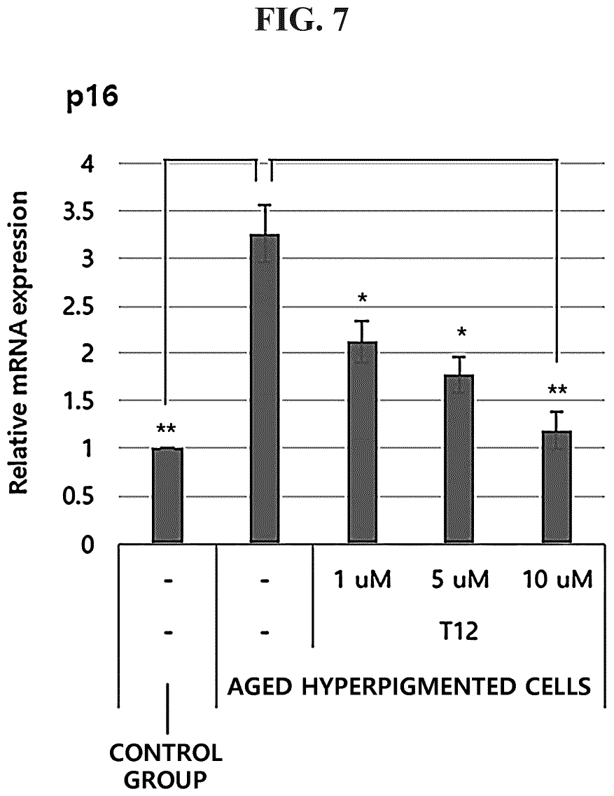

Furthermore, with reference to FIG. 7, the inhibitory efficacy of T12 on the p16 gene expression was confirmed.

This experiment was conducted by post-treating the test material on cells in which melanin production had been excessively induced; therefore, the experimental results are reasonably interpreted as melanin decomposition effects. In addition, the melanin production inhibitory effect is considered to result from inhibition of melanin-producing enzyme expression, thereby inhibiting new melanin production, and this interpretation is supported by the TRP2 analysis results.

What is claimed is:

1. A method for skin anti-aging, skin brightening, or skin reverse-aging, comprising:
administering to a subject in need thereof an effective amount of a composition comprising, as an active ingredient, a compound represented by Chemical Formula 1, a stereoisomer thereof, a pharmaceutically acceptable salt thereof, a hydrate thereof, or a solvate thereof, 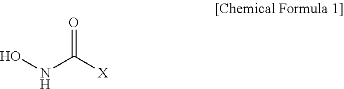
in Chemical Formula 1,
X is represented by any one of Chemical Formulae 1-1 to 1-5 below, 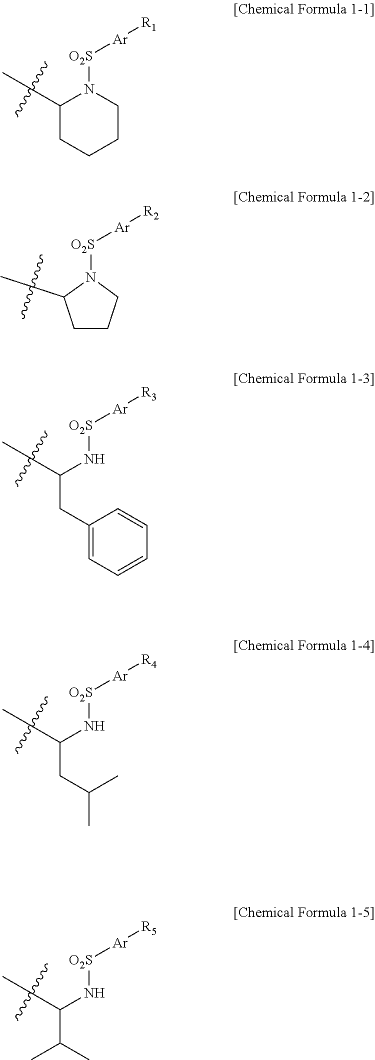
in Chemical Formula 1-1,
Ar is a phenyl which is unsubstituted or substituted with one to three Ri groups,
R₁ is hydrogen, phenyl group, C1-C4 alkoxy group, C1-C3 alkyl group, fluoro group, adamantyl group, acetylamino group, or hydroxyiminoethyl group, and
when the number of R₁ residues is more than one, all R₁ residues are identical to each other,
in Chemical Formula 1-2,
Ar is a phenyl substituted with one R₂, and
R₂ is C1-C4 alkyl group, C1-C4 alkoxy group, adamantyl group, acetyl group, or acetylamino group,
in Chemical Formula 1-3,
Ar is a phenyl substituted with one to three R₃ groups or an unsubstituted naphthalene,
R₃ is hydrogen or a C1-C4 alkyl group, and
when the number of R₃ residues is more than one, all R₃ residues are identical to each other, and preferably, R₃ is a C1-C4 alkyl group,
in Chemical Formula 1-4,
Ar is a phenyl which is unsubstituted or substituted with one to three R₄ groups,
R₄ is hydrogen, phenyl group, C1-C4 alkyl group, or C1-C4 alkoxy group, and
when the number of R₄ residues is more than one, all R₄ residues are identical to each other, and
in Chemical Formula 1-5,
Ar is a phenyl substituted with one to three R₅ groups,
R₅ is a C1-C4 alkyl group, and
when the number of R₅ residues is more than one, all R₅ residues are identical to each other.

2. The method of claim 1, wherein the compound represented by Chemical Formula 1 is any one selected from the following compounds:
N-hydroxy-1-(phenylsulfonyl)piperidine-2-carboxamide,
1-([1,1′-biphenyl]-4-ylsulfonyl)-N-hydroxypiperidine-2-carboxamide,
1-((4-butoxyphenyl)sulfonyl)-N-hydroxypiperidine-2-carboxamide,
N-hydroxy-1-(mesitylsulfonyl)piperidine-2-carboxamide,
2-((4-(tert-butyl)phenyl)sulfonamido)-N-hydroxy-3-phenylpropanamide,
N-hydroxy-3-phenyl-2-((2,4,6-trimethylphenyl)sulfonamido)propanamide,
N-hydroxy-2-(naphthalen-2-sulfonamido)-3-phenylpropanamide,
2-([1,1′-biphenyl]-4-sulfonamido)-N-hydroxy-4-methylpentanamide,
2-((4-(tert-butyl)phenyl)sulfonamido)-N-hydroxy-4-methylpentanamide,
2-((4-butoxyphenyl)sulfonamido)-N-hydroxy-4-methylpentanamide,
N-hydroxy-4-methyl-2-((2,4,6-trimethylphenyl)sulfonamido)pentanamide,
2-((4-(tert-butyl)phenyl)sulfonamido)-N-hydroxy-3-methylbutanamide,
N-hydroxy-3-methyl-2-((2,4,6-trimethylphenyl)sulfonamido)butanamide,
1-((4-fluorophenyl)sulfonyl)-N-hydroxypiperidine-2-carboxamide,
1-((4-((3r,5r,7r)-adamantan-1-yl)phenyl)sulfonyl)-N-hydroxypiperidine-2-carboxamide,
N-hydroxy-1-((4-propylphenyl)sulfonyl)piperidine-2-carboxamide,
1-((4-acetamidophenyl)sulfonyl)-N-hydroxypiperidine-2-carboxamide,
1-((4-(tert-butyl)phenyl)sulfonyl)-N-hydroxypyrrolidine-2-carboxamide,
1-((4-butoxyphenyl)sulfonyl)-N-hydroxypyrrolidine-2-carboxamide,
1-((4-((3r,5r,7r)-adamantan-1-yl)phenyl)sulfonyl)-N-hydroxypyrrolidine-2-carboxamide,
N-hydroxy-1-((4-propylphenyl)sulfonyl)pyrrolidine-2-carboxamide,
1-((4-acetylphenyl)sulfonyl)-N-hydroxypyrrolidine-2-carboxamide,
N-hydroxy-1-((4-(1-(hydroxyimino)ethyl)phenyl)sulfonyl)piperidine-2-carboxamide,
N-hydroxy-4-methyl-2-(phenylsulfonamido)pentanamide, and
1-((4-acetamidophenyl)sulfonyl)-N-hydroxypyrrolidine-2-carboxamide.

3. The method of claim 1, wherein the compound represented by Chemical Formula 1 is any one selected from the following compounds:
N-hydroxy-1-(phenylsulfonyl)piperidine-2-carboxamide,
1-([1,1′-biphenyl]-4-ylsulfonyl)-N-hydroxypiperidine-2-carboxamide,
N-hydroxy-2-(naphthalen-2-sulfonamido)-3-phenylpropanamide,
2-([1,1′-biphenyl]-4-sulfonamido)-N-hydroxy-4-methylpentanamide,
2-((4-(tert-butyl)phenyl)sulfonamido)-N-hydroxy-4-methylpentanamide,
2-((4-(tert-butyl)phenyl)sulfonamido)-N-hydroxy-3-methylbutanamide, and
N-hydroxy-1-((4-propylphenyl)sulfonyl)pyrrolidine-2-carboxamide.

4. The method of claim 1, wherein the compound represented by Chemical Formula 1 is derived from an amino acid.

5. The method of claim 1, wherein the method restores aged melanin-producing cells to young melanin-producing cells.

6. The method of claim 1, wherein the method inhibits a gene expression of one or more of P21 and P16.

7. The method of claim 1, wherein the method inhibits melanin production.

8. The method of claim 1, wherein the method inhibits mRNA expression of Tyrosinase-related protein 2.

9. The method of claim 1, wherein the composition is a cosmetic composition.

10. The method of claim 1, wherein the composition is for skin external application.

11. The method of claim 1, wherein the composition is a food composition.

12. The method of claim 1, wherein the composition is a pharmaceutical composition.

13. The method of claim 12, wherein the pharmaceutical composition is for preventing, improving, or treating skin hyperpigmentation.

14. The method of claim 1, wherein content of the active ingredient is 0.01 μM to 100 mM based on a total volume of the composition.

15. The method of claim 1, wherein a daily application amount of the active ingredient is 1 to 1000 mg/kg.

16. The method of claim 1, wherein the active ingredient is treated to cells at a concentration of 1 to 100 μM.
